(19) United States
(12) Patent Application Publication
(10) Pub. No.: US 2021/0238302 A1
(43) Pub. Date: Aug. 5, 2021

(54) HIGH AFFINITY MONOCLONAL ANTIBODIES (MABS) AGAINST CELL SURFACE EXPRESSED HUMAN CARBONIC ANHYDRASE IX (HCA-IX), AND USES THEREOF

(71) Applicant: NATIONAL RESEARCH COUNCIL OF CANADA, Ottawa (CA)

(72) Inventors: Anne E. G. LENFERINK, Lorraine (CA); Maureen D. O'CONNOR, Beaconsfield (CA)

(21) Appl. No.: 17/050,620

(22) PCT Filed: Apr. 26, 2019
(86) PCT No.: PCT/CA2019/050540

§ 371 Date: Oct. 26, 2020

Related U.S. Application Data
(60) Provisional application No. 62/663,662, filed on Apr. 27, 2018.

(51) Int. Cl. C07K 16/30; G01N 33/573

(52) U.S. Cl. CPC C07K 16/30; G01N 33/573; G01N 2333/988; C07K 2317/92; C07K 2317/34; C07K 2317/76; C07K 2317/77; C07K 2317/24

(57) ABSTRACT

The present invention provides a set of Carbonic Anhydrase-IX monoclonal antibodies (CA-IX mAbs) that bind with high affinity to cell-surface expressed hCA-IX and has enzyme inhibiting characteristics. These mAbs have the potential to become the next biologics for the treatment of renal and possibly other types of cancer.

### SEQUENCE LISTING

The present application contains a Sequence Listing which has been submitted electronically in ASCII format and is hereby incorporated by reference in its entirety. Said ASCII copy, created on Apr. 18, 2019, is named “2019-04-18 Sequence Listing_ST25.txt” and is 36 Kbites in size.

### FIELD OF THE INVENTION

The present invention relates to monoclonal antibodies (CA-IX mAbs) that bind with high affinity to cell-surface expressed human Carbonic Anhydrase-IX (hCA-IX) and have enzyme-inhibiting characteristics.

### BACKGROUND OF THE INVENTION

Carbonic anhydrases (CA) are a family of 16 distinct but related metalloenzymes which catalyze, albeit with various activity (FIG. 1), the reversible hydration of carbon dioxide (CO₂) to bicarbonate (HCO₃⁻) and protons (H⁺) (Svastova et al., 2003, Mboge et al., 2018). CAs can be found in many human organs, tissues and subcellular compartments where they play an important role in the regulation of the extracellular and intracellular pH (pHe and pHi, respectively), and the secretion of electrolytes (Zatovicova et al., 2005; Thiry et al., 2006). Two of the CA family members: CA-IX and CA-XII have been associated with cancer progression, metastasis and reduced therapeutic response (Neri et al., 2011).

CA-IX (also known as MN, or RCC-associated protein G250), is like CA-XII, a transmembrane protein with an extracellular catalytic site, but unlike CA-XII, CA-IX has an NH₂-terminal proteoglycan (PG)-like domain. The C-terminal intracellular portion of CA-IX is involved in the inside-out regulation of the extracellular catalytic domain through the phosphorylation of Thr-443 by protein kinase A (PKA) (Hulikova et al., 2009; Ditte et al., 2011). In addition to its pH-balancing activities, CA-IX has been shown to be involved in cell adhesion and migration (Svastova et al., 2012).

Expression of CA-IX is tightly controlled by hypoxia-inducible factor 1 alpha (HIF-1α). CA-IX is expressed on the cell surface of tumor cells located in pre-necrotic areas tumor (Wykoff et al., 2000) where it is involved in the accelerated degradation of the extracellular matrix (ECM) while promoting tumor cell survival and metastasis.

The selective cancer specific expression of CA-IX, especially in Clear Cell Renal Cell Carcinoma (CCRCC), and its restricted expression in normal tissue, makes CA-IX a very attractive therapeutic target (Pastorekova et al., 2004; Zavada et al., 2000).

The most prominent mAbs raised against CA-IX are the M75 mAb (Zavada et al., 1993), which binds to CA-IX's PG-like domain, and the G250 mAb (Oosterwijk et al., 1986), which interacts with CA-IX's catalytic domain without inhibiting its enzyme activity.

M75 mAb has mostly been used as tool for CA-IX detection (Chrastina et al., 2003a, 2003b; Zatovicova et al., 2010) but also in a functional nanoparticle format (Antal et al., 2018) whereas the chimeric version of G250, aka cG250, was further developed as a therapeutic mAb (Surfus et al., 1996; Oosterwijk, 2008). Studies using the cG250 indicated that the modus operandi of this mAb is an Antibody-Dependent Cellular Cytotoxicity (ADCC) response. cG250 (aka Girentuximab) is currently commercialized by WILEX AG under the name RENCAREX®. In late 2012, WILEX AG reported however that the cG250 mAb did not improve the disease-free survival rate of patients (>6-year span) compared to a placebo in clinical trails and announced that their late-stage trial, called ARISER, was to be terminated. Nonetheless, the Iodine (124I) radiolabelled Girentuximab (REDECTANE®) is continued to be developed as an imaging diagnostic agent for the detection CCRCC. A Phase III trial has shown that REDECTANE® in conjunction with PET/CT imaging is superior to a CT scan alone in the diagnosis of CCRCC. Lastly, Nuclea who recently acquired WILEX Inc., a subsidiary of WILEX AG, is developing a CA-IX in vitro diagnostics (IVD) immunohistochemistry (IHC) assay (named “CAIX Dx”). This assay will be used for patient stratification in a planned study with RENCAREX® and as a potential future companion diagnostic in the adjuvant treatment of CCRCC.

Other clinical trials with cG250 have been using the mAb as an adjuvant for the treatment of metastatic RCC in surgically-treated RCC patients with a high risk of relapse, and as a combination therapy with Interleukin-2 (IL-2) or Interferon-α (IFN-α) (Zatovicova et al., 2010; Neri et al., 2011; Siebels et al., 2011). In addition, cG250 has been labeled with a variety of radionuclides (¹²⁴I, ¹¹¹In, ⁸⁹Zr, ¹³¹I, ⁹⁰Y, and ¹⁷⁷Lu) and is one the most explored CA-IX radiopharmaceuticals (Lau et al., 2017; Brouwers et al. 2004; Stillebroer et al., 2012).

Lately it was reported that WILEX AG also initiated the development of a cG250 antibody drug conjugate (ADC) by its subsidiary Heidelberg Pharma GmbH. This subsidiary signed a license agreement with Roche for the joint development of a novel class of ADCs based on Heidelberg Pharma's patented technology (Antibody Targeted Amanitin Conjugates, ATACs). In addition to cG250, other anti-CA-IX mAbs have been generated with the goal to deliver cytotoxic agents or radionuclides into CA-IX expressing tumor cells. E.g. Petrul et al. (2012) isolated the 3ee9 Fab by panning recombinant human CA-IX extracellular domain (ECD) against a library of human Fabs. This Fab engineered into a mAb was further developed as an ADC by BAYER Healthcare (BAY79-4620) by conjugation to monomethyl auristatin E (MMAE). BAY79-4620 showed potent antitumor efficacy and a Phase I clinical trial to determine the maximal tolerated dose (MTD) was recently completed by BAYER Healthcare.

To target the CA-IX catalytic domain, several small molecule inhibitors such as the sulfonamide/sulfamate- and coumarin-based compounds (Supuran et al., 2008; Neri et al., 2011; Pacchiano et al., 2010; Lou et al., 2011) have been shown to effectively inhibit the enzymatic activity of CA-IX. Small molecules often lack target specificity though, and recent efforts have focused on using phage display libraries to identify novel anti-CA-IX antibodies (mAbs) that target CA-IX's catalytic domain (Alshkog et al., 2009; Xu et al, 2010; Murri-Plesko et al., 2011). Several of these Abs displayed in vitro CA-IX enzyme inhibiting activities but in vivo experiments were not performed. An exception is the VII/20 mAb (hybridoma-derived) which demonstrated in vitro enzyme inhibiting activity and in vivo tumor growth inhibition of freshly inoculated HT-29 colorectal tumor cells, however only limited effects were reported on established tumors (Zatovicova et al., 2010). There is much evidence that targeting CA-IX's enzyme activity interferes with its role in the pH regulation in cancer cells. Recent studies have shown that CA-IX activates many signalling mechanisms that appear to influence the response of cancer cells to radiation therapy (Ward et al., 2018).

Given its specific tumor expression, CA-IX is a ‘hot’ target; several clinical trials targeting CA-IX using modalities, such as small molecules and protein-based therapeutics, either alone or in combination therapies have been carried out or are on-going. The cG250 mAb (Girentuximab; trade name RENCAREX®) has been commercialized by WILEX AG (name change to Heidelberg Pharma AG in 2017).

### SUMMARY OF THE INVENTION

Therefore, the present invention provides a monoclonal antibody that bind to CA-IX's catalytic domain and/or inhibits its catalytic activity in vitro and in cellular assays. These MAbs have the potential to become the next biologics for the treatment of renal and possibly other types of cancer.

Presently, two novel antibodies (4A2 and 9B6) have been identified that specifically inhibit the catalytic activity of human CA-IX.

Therefore, in a first aspect, the present invention provides a monoclonal antibody or a fragment thereof, that specifically binds to Carbonic Anhydrase-IX (CA-IX) catalytic domain.

In a second aspect, the antibody or fragment specifically binds to a peptide selected from the group consisting of: DQSHW (SEQ ID NO.2), DEALGR (SEQ ID NO.3), STAFARVDE (SEQ ID NO.4), and STAFARVDEALGR (SEQ ID NO.5). Particularly, the antibody or fragment specifically binds to a peptide selected from the group consisting of: DEALGR (SEQ ID NO.3), STAFARVDE (SEQ ID NO.4), and STAFARVDEALGR (SEQ ID NO.5)

According to a further aspect, the antibody of fragment inhibits the catalytic activity of CA-IX in an enzymatic assay and in cellular assay.

According to a further aspect, the antibody or fragment inhibits CA-IX by at least 25%, when used at a concentration of 0.5 μM in an in vitro enzymatic assay.

Still in a further aspect, the antibody or fragment inhibits CA-IX by at least 20% when used at a concentration of 0.5 μM in an in vitro cellular assay.

The present invention further provides an antibody or fragment thereof which comprises a light chain comprising: a complementarity determining region (CDR) L1 comprising the sequence X₁ASX₂SVX₃X₄X₅X₆X₇X₈YMX₉ wherein X₁ is S or K, X₂ is S or Q, X₃ is D or no amino acid, X₄ is Y or no amino acid, X₅ is D or no amino acid, X₆ is G or no amino acid, X₇ is N or no amino acid, X₈ is G or S, X₉ is H or N (SEQ ID NO.32); a CDR L2 comprising the sequence X₁₀X₁₁SX₁₂LX₁₃S wherein X₁₀ is D or E, Xu is T or A, X₁₂ is N or S, X₁₃ is S or E (SEQ ID NO.33); and a CDR L3 comprising the sequence QQX₁₄X₁₅X₁₆X₁₇PX₁₈T wherein X₁₄ is W or S, X₁₅ is R or Y, X₁₆ S or E, X₁₇ Y or G and PX₁₈ is P or Y (SEQ ID NO.34); or a sequence substantially identical thereto; wherein the antibody or fragment is specific for CA-IX.

The present invention further provides an antibody or fragment thereof which comprises a heavy chain comprising: a complementarity determining region (CDR) H1 comprising a peptide defined by sequence: GX₂₁X₂₂FX₂₃X₂₄X₂₅WX₂₆X₂₇ wherein X₂₁ is F or Y, X₂₂ is T or I, X₂₃ is S or T, X₂₄ is Y or T, X₂₅ is Y or K, X₂₆ is M or I, X₂₇ is D or N (SEQ ID NO.35); a CDR H2 comprising a peptide defined by sequence: EIRLKSDNYATHY AESVKGA (SEQ ID NO.36); and a CDR H3 comprising a peptide defined by sequence: PHYYGYFDY (SEQ ID NO.37); or a sequence substantially identical thereto; wherein the antibody or fragment is specific for CA-IX.

Still in a further aspect, there is provided a composition comprising the antibody 4A2 as defined herein, in combination with the antibody 9B6 as defined herein.

Still in a further aspect, there is provided a composition comprising one or more than one antibody or fragment as defined herein, in admixture with a pharmaceutically-acceptable carrier, diluent, or excipient.

Still in a further aspect, there is provided a method of inhibiting Carbonic anhydrase-IX (CA-IX) enzymatic activity in a cell, comprising contacting the cell with the antibody or fragment as defined herein, optionally linked to a chemotherapeutic drug.

Still in a further aspect, there is provided a method of preventing or treating cancer in a subject, comprising administering a pharmaceutically acceptable dose of an antibody or fragment as defined herein, the subject.

The present invention further provides the use of the antibody or fragment as defined herein, as a companion diagnostic in the adjuvant treatment of cancer.

The present invention further provides the antibody or fragment as defined herein, for the manufacture of a composition for the treatment or prevention of cancer in a subject.

The present invention further provides the antibody or fragment as defined herein, for use in the treatment or prevention of cancer in a subject.

Still, the present invention further provides an antigen-binding fragment that specifically binds to a 6 to 12 amino acid peptide comprised within the epitope STAFARVDEALGR (SEQ ID NO. 5).

The present invention also provides a nucleic acid molecule encoding the isolated or purified antibody or fragment thereof as described herein.

A vector comprising the nucleic acid molecule as just described is also provided.

The isolated or purified antibody or fragment thereof as described herein may be immobilized onto a surface or may be linked to a cargo molecule. The cargo molecule may be a detectable agent, a therapeutic agent, a drug, a peptide, an enzyme, a growth factor, a cytokine, a receptor trap, an antibody or fragment thereof (e.g., IgG, scFv, Fab, VHH, etc) a chemical compound, a carbohydrate moiety, DNA-based molecules (anti-sense oligonucleotide, microRNA, siRNA, plasmid), a cytotoxic agent, viral vector (adeno-, lenti-, retro-), one or more liposomes or nanocarriers loaded with any of the previously recited types of cargo molecules, or one or more nanoparticle, nanowire, nanotube, or quantum dots. In a specific, non-limiting example, the cargo molecule is a cytotoxic agent.

The present invention further provides an in vitro method of detecting CA-IX is also provided, the method comprising: a) contacting a tissue sample with one or more than one isolated or purified antibody or fragment thereof as described herein linked to a detectable agent; and b) detecting the detectable agent linked to the antibody or fragment thereof bound to CAIX in the tissue sample.

In the method described above, the method may detect CA-IX in circulating cells and the sample may be a serum sample. In the method as described, the step of detecting (step b) may be performed using optical imaging, immunohistochemistry, molecular diagnostic imaging, ELISA, or other suitable method.

The present invention further provides an in vivo method of detecting CA-IX expression in a subject, comprising: a) administering one or more than one isolated or purified antibody or fragment thereof as described herein linked to a detectable agent to the subject; and b) detecting the detectable agent linked to the antibody or fragment thereof bound to CA-IX.

In the method described just described, the step of detecting (step b)) is performed using PET, SPECT, fluorescence imaging, or any other suitable method.

Additional aspects and advantages of the present invention will be apparent in view of the following description. The detailed descriptions and examples, while indicating preferred embodiments of the invention, are given by way of illustration only, as various changes and modifications within the scope of the invention will become apparent to those skilled in the art, in light of the teachings of this invention.

### DESCRIPTION OF THE INVENTION

### Description of the Figures

FIG. 1: Cartoon showing the domains, subcellular localization and catalytic activity of the human (h) carbonic anhydrase (CA) family. The cytoplasmic and mitochondrial hCA-I, -II, -III, -VII, -VIII, -X, -XI and -XIII are composed of only a catalytic domain, whereas the secreted hCA-VI has a short C-terminal domain, and the membrane-associated hCA-IV, -VI, -IX, -XII, and -XIV have a transmembrane anchor and, except hCA-IV, also a cytoplasmic tail. hCA-IX is the only human Carbonic Anhydrase that displays an N-terminal proteoglycan (PG) sequence, which is involved in the cell-cell adhesion process (Adapted from Pastorekova et al., 2004).

FIG. 2: Coomassie Brilliant Blue stained SDS-PAGE of the presently-produced rhCA-IX ECD under reducing and non-reducing conditions. The disulphide-bonded dimer rhCA-IX dimer has a molecular weight of ˜110 kDa, whereas the monomer and the reduced dimer are ˜48 kDa.

FIGS. 3A-B: Non-purified CA-IX mAbs (undiluted CM) were evaluated by western blot for binding to the purified CA-IX ECD antigen. mAbs 4A2 and 9B6 failed to bind to rhCA-IX ECD under both non-reducing (A) and reducing conditions (B), respectively. Anti-hCA-IX mAb 10F2 is shown as a positive control.

FIGS. 4A-D: SEC profile of the hCA-IX ECD produced in CHO cells shows the presence of monomers and dimers (A). Monomer and dimer containing fractions were re-evaluated by SEC after storage for 2 weeks at 4° C. (B; monomer; C: dimer; D: overlay of B and C).

FIGS. 5A-B: Epitope mapping of the mAbs 4A2 and 9B6 using the PepScan technology (www.pepscan.com). (A) Projection of the mAb 4A2 and 9B6 putative binding epitope sequences as determined by the PepScan analysis on the known X-ray structure of the hCA-IX catalytic domain (in silico; see Alterio et al, 2009) implies that mAb 4A2 and 9B6 bind to distinct epitopes within this domain. (B) Location of these epitopes in hCA-IX linear amino acid sequence: 4A2 binding epitope is indicated as underlined in bold (DQSHW and DEALGR), the 9B6 epitope is depicted in bold (STAFARVDE), whereas the two amino acid (DE) overlap between the 4A2 epitope (DEALGR) and the 9B6 epitope (STAFARVDE) is indicated in bold underlined italics (STAFARVDEALGR).

FIGS. 6A-B: SPR binding competition experiment of the 4A2 and 9B6 mAbs by Surface Plasmon Resonance (SPR). (A) depicts the principle of the binding assay. (B) Color coded ‘checker board’ representation of the results of the SPR binding competition experiment showing that mAbs 4A2 and 9B6 do not compete for binding (dark boxes) when either using the rhCA-IX ECD monomer or dimer.

FIG. 7A-B: Evaluation of the enzyme inhibiting attributes of mAbs 4A2 and 9B6. (A) The rhCA-IX ECD (1 μM, mixture) is catalytically active and can be fully inhibited by 10 μM of the small molecule inhibitor Acetozolamide. (B) Both 4A2 and 9B6 inhibit the rhCA-IX ECD enzyme activity by 61.94% and 42.59% respectively; the dotted line indicates 100% CA-IX catalytic activity. Displayed are the average values+SEM of a duplicate experiment.

FIGS. 8A-B: SK-RC-59 and SK-RC-52 cells have been described as respectively high and low hCA-IX expressing (Ebert et al., 1990). SDS-PAGE evaluation (whole cell lysate) of hCA-IX expression (★) in these cell lines under reducing (A) and non-reducing (B) conditions of these cell lines.

FIGS. 9A-B: Evaluation of the CA-IX mAbs 4A2 and 9B6 for binding by flow cytometry to their cognate target expressed by the human renal carcinoma SK-RC-52 (A; high hCA-IX) and SK-RC-59 (A; low hCA-IX) cell lines. The M75 mAb (Zavada et al., 1993) and the commercial hCA-IX mAb2188) were used as positive controls, the secondary mAb alone (2^{nd}) was used to evaluate non-specific signals. Shown is the % of live cells in each of the experiments (left Y-axis) and the mean fluorescent intensity that results from mAb binding to cell surface expressed hCA-IX (right Y-axis).

FIGS. 10A-B: CDR1-3 sequence alignment of the mAb 4A2 and 9B6 VL (A) and V_{H} (B) region using the MUSCLE 3.7 web interface (phylogeny.lirmm.fr/phylo_cgi/index.cgi). Consensus symbols: * (asterisk), single, fully conserved residue; : (colon), conservation between groups of strongly similar properties; scoring >0.5 (Gonnet PAM 250 matrix); . (period), conservation between groups of weakly similar properties; scoring=<0.5 (Gonnet PAM 250 matrix; From: Dereeper et al., 2010. Dereeper et al., 2008. Edgar, 2004).

FIG. 11: SDS-PAGE of the 4A2 and c9B6 mAbs expressed recombinantly (mouse IgG2b format) in CHO cells using a 1:1 V_{L}:V_{H} ratio in a small-scale (50 mL) expression experiment. Conditioned medium was harvested on day 7, ProtA purified, and quantitated. Both the conditioned medium (CM) and ProtA purified chimeric mAbs (P) were evaluated (non-reducing conditions).

FIG. 12: SPR experiments using the recombinant ProtA purified 4A2 and 9B6 mAbs. Briefly, the 4A2 and 9B6 mAbs were captured on the chip surface with an anti-mouse Fc antibody. Flowing of purified rhCA-IX ECD dimer at various concentrations indicates that the binding characteristics of 4A2 and 9B6 are very different.

FIG. 13: Real time SPR binding results of the 4A2 and 9B6 mAbs to rhCA-IV, rhCA-XII, rhCA-XIV, and rmCA-IX, showing the hCA-IX specificity of these mAbs. Murine anti-hCA-XIV was used as a positive control for the hCA-XIV surface (dark boxes: no binding; light boxes: binding).

FIGS. 14A-B: Results of the thermostability experiments, using the DSC, of mAbs 4A2 and 9B6 (dashed line) in comparison to the therapeutic antibody Cetuximab (anti-EGFR Ab; solid line). The thermostability of mAbs 4A2 is very similar to that of 9B6, however both mAbs are less thermostable than the Cetuximab control.

FIGS. 15A-B: Epitope mapping of the 4A2 and 9B6 mAbs by Yeast Surface Display (YSD). (A) Cartoon depicting the antigen fragments presented on the surface of yeast cell (Adapted from Feldhaus et al., 2003). (B) Nine (9) peptides covering the entire hCA-IX ECD were expressed on the yeast membrane to map the binding region of the 4A2 and 9B6 mAbs.

FIGS. 16A-C: Binding competition experiment by SPR. mAbs 4A2 (A), 9B6 (B) and cG250 (C) were directly immobilized on the SPR chip (‘mAb1’). Then the CA-IX monomer followed by either one of these mAbs (‘mAb2’) was flowed over the ‘mAb1’ surface using the co-inject method. These results not only confirm that the 4A2 and 9B6 mAbs do not compete for binding to hCA-IX but also shows that neither mAb competes with the cG250 mAb, indicating that all three mAbs bind to separate hCA-IX epitopes.

FIGS. 17A-B: Evaluation of the enzyme inhibiting attributes of the 4A2 and 9B6 mAbs (underlined) and their Fabs, either alone or in combo. (A) Incubation of the rhCA-IX ECD (0.5 μM dimer) with the 4-NPA fluorescent substrate shows that the hCA-IX protein is catalytically active (substrate alone) and that its activity can be fully inhibited by 10 μM Acetozolamide (substrate+SMI). The 4A2 and 9B6 mAb or their respective Fabs inhibit rhCA-IX ECD enzyme activity, albeit to various degrees (4A2 mAb=40%, 9B6 mAb=28%, 4A2+9B6mAb=50%, 4A2Fab=22%, 9B6Fab=24%, 4A2+9B6 Fab=32%). (B) Additional experiments using 67NR/hCA-IX cells and hCA-IX natural substrate (CO₂) shows that the 4A2 mAb (100 μg/mL) also inhibits cellularly expressed hCA-IX activity (49%), while a non-specific control (NS-mIgG) and a specific CA-IX control mAb (CA-IX mIgG CTL) do not inhibit. CA-IX/CA-XII small molecule inhibitor U104 (50 nM) was used as positive control.

FIGS. 18A-B: Evaluation of the internalization of mAb 4A2 and 9B6. Both mAbs were labeled with a pH sensitive dye (pH-Ab) which becomes fluorescent at lower pH, hence when the mAbs enter in the endosomes. Both are internalized and specifically accumulate intracellularly in hCA-IX expressing SK-RC-52 cells after 24 h of incubation at 37° C. (A). In a second experiment, cells coated with serial dilutions of the mAbs (30 min, 4° C.), washed and then transferred to 37° C., shows that 4A2 accumulates at a faster rate than 9B6 (B).

### ABBREVIATIONS

ADC: antibody drug conjugate; ADCC: antibody-dependent cellular cytotoxicity; CCRCC: Clear Cell Renal Cell Carcinoma; CDR: Complementarity determining region; ECD: extracellular domain; ELISA: Enzyme-Linked ImmunoSorbent Assay; mAb(s): monoclonal antibody(ies); RPPA: Reverse Phase Protein Array; Surface Plasmon Resonance (SPR).

### Definitions

The term “about” as used herein refers to a margin of + or −10% of the number indicated. For sake of precision, the term about when used in conjunction with, for example: 90% means 90%+/−9% i.e. from 81% to 99%. More precisely, the term about refer to + or −5% of the number indicated, where for example: 90% means 90%+/−4.5% i.e. from 86.5% to 94.5%. When used in the context of a pH, the term “about” means+/−0.5 pH unit.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

As used herein, the terms “disease” may be used interchangeably or may be different in that the particular disorder, infection or condition may not have a known causative agent (so that etiology has not yet been worked out) and it is therefore not yet recognized as a disease but only as an undesirable condition or syndrome, wherein a more or less specific set of symptoms have been identified by clinicians.

The term “subject” as used herein refers to an animal, preferably a mammal or a bird, who is the object of administration, treatment, observation or experiment. “Mammal” includes humans and both domestic animals such as laboratory animals and household pets, (e.g. cats, dogs, swine, cattle, sheep, goats, horses, rabbits), and non-domestic animals such as wildlife, fowl, birds and the like. More particularly, the mammal is a rodent. Still, most particularly, the mammal is a human.

The molecule(s) described herein can be formulated as pharmaceutical compositions by formulation with additives such as pharmaceutically acceptable excipients, pharmaceutically acceptable carriers, and pharmaceutically acceptable vehicles.

As used herein, the term “pharmaceutically acceptable” refers to molecular entities and compositions that are physiologically tolerable and do not typically produce an allergic or similar unwanted reaction, such as gastric upset, dizziness and the like, when administered to human. Preferably, as used herein, the term “pharmaceutically acceptable” means approved by regulatory agency of the federal or state government or listed in the U.S. Pharmacopeia or other generally recognized pharmacopeia for use in animals, and more particularly in humans.

The term “adjuvant” refers to a diluent, excipient, or vehicle with which the compounds of the present invention may be administered. Sterile water or aqueous saline solutions and aqueous dextrose and glycerol solutions may be employed as carrier, particularly for injectable solutions. Suitable pharmaceutical carriers are described in “Remington's Pharmaceutical Sciences” by E.W. Martin (1966).

If administered as a medicinal preparation, the composition can be administered, either as a prophylaxis or treatment, to a patient by a number of methods. The present compositions may be administered alone or in combination with other pharmaceutical agents and can be combined with a physiologically acceptable carrier thereof. The effective amount and method of administration and aim of the present formulation can vary based on the individual subject, the stage of the disease or condition, and other factors apparent to one skilled in the art. In the case of a pharmaceutical formulation, during the course of the treatment, the concentration of the present compositions may be monitored (for example, blood antibody levels may be monitored) to ensure that the desired response is obtained.

### DETAILED DESCRIPTION OF PARTICULAR EMBODIMENTS

Presently, two novel antibodies (4A2 and 9B6) have been identified that specifically inhibit human CA-IX catalytic activity. The combination of these two monoclonal antibodies (4A2 and 9B6) was shown to be synergistic over the inhibiting activity of each, indicating that each antibody is directed towards a different epitope.

It was also shown that the combination of these two monoclonal antibodies (4A2 and 9B6) provided inhibitory activity of the enzyme is synergistic over the respective inhibiting activity of each, further providing indications that each antibody is directed towards a different epitope linked to the catalytic activity of the enzyme. It was also shown that mAb 4A2 and 9B6 were active in reducing cell viability, although slightly less active than the M75 mAb (a known antibody) which does not inhibit the catalytic activity in vivo.

The present invention relates to Carbohydrate Anhydrase IX-specific antibodies, fragments thereof, and uses thereof. More specifically, the present invention relates to enzyme-inhibiting Carbohydrate Anhydrase IX-specific antibodies and fragments thereof and their use for the treatment of CA-IX associated diseases or disorders.

### Antibodies

In a particular embodiment, there is therefore provided an antibody or fragment thereof, wherein the antibody or fragment has enzyme inhibiting activity against Carbohydrate Anhydrase IX.

In a further embodiment, the antibody or fragment binds to Carbonic Anhydrase-IX (CA-IX) catalytic domain.

Particularly, the antibody of fragment inhibits CA-IX catalytic activity in enzymatic assay and cellular assays. More particularly, the antibody or fragment inhibits CA-IX by at least 25%, when used at a concentration of 0.5 μM in an enzymatic assay. Still, particularly, the antibody or fragment inhibits hCA-IX by at least 20% when used at a concentration of 0.5 μM in a cellular assay.

In accordance with a particular embodiment, the antibody or fragment is isolated or purified.

In a particular embodiment, the antibody or fragment is used for the manufacture of a composition for the treatment or prevention of cancer in a subject. More particularly, the cancer is a hCA-IX expressing cancer, such as, but not limited to, Clear Cell Renal Cell Carcinoma (CCRCC) in humans. The antibody or fragment thereof as defined in any one of claims 1 to 27, for use in the treatment or prevention of cancer in a subject.

The term “antibody”, also referred to in the art as “immunoglobulin” (Ig), as used herein refers to a protein constructed from paired heavy and light polypeptide chains; various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (VL) and a constant (CL) domain, while the heavy chain folds into a variable (VH) and three constant (CH, CH2, CH3) domains. Interaction of the heavy and light chain variable domains (VH and VL) results in the formation of an antigen binding region (Fv). Each domain has a well-established structure familiar to those of skill in the art.

The isolated or purified antibody or fragment thereof as described herein may a full-length IgG, Fv, scFv, Fab, or F(ab′)2; the antibody or fragment thereof may also comprise framework regions from IgA, IgD, IgE, IgG, or IgM. The isolated or purified antibody or fragment thereof of the present invention may be chimeric; for example, and without wishing to be limiting, such a chimeric antibody or fragment thereof may comprise the VL and VH domains from mouse and framework regions (constant domains) from human IgG1, more specifically human kappa 1 light chain and human IgG1 heavy chain.

The light and heavy chain variable regions are responsible for binding the target antigen and can therefore show significant sequence diversity between antibodies. The constant regions show less sequence diversity and are responsible for binding a number of natural proteins to elicit important biochemical events. The variable region of an antibody contains the antigen-binding determinants of the molecule, and thus determines the specificity of an antibody for its target antigen. Most of the sequence variability occurs in six hypervariable regions, three each per variable heavy (VH) and light (VL) chain; the hypervariable regions combine to form the antigen-binding site and contribute to binding and recognition of an antigenic determinant. The specificity and affinity of an antibody for its antigen is determined by the structure of the hypervariable regions, as well as their size, shape, and chemistry of the surface they present to the antigen. Various schemes exist for identification of the regions of hypervariability, the two most common being those of Kabat and of Chothia and Lesk. Kabat et al. (1991) define the “complementarity-determining regions” (CDR) based on sequence variability at the antigen-binding regions of the VH and VL domains. Chothia and Lesk (1987) define the “hypervariable loops” (H or L) based on the location of the structural loop regions in the VH and VL domains. As these individual schemes define CDR and hypervariable loop regions that are adjacent or overlapping, those of skill in the antibody art often utilize the terms “CDR” and “hypervariable loop” interchangeably, and they may be so used herein. A more recent scheme is the IMGT numbering system (Lefranc et al., 2003), which was developed to facilitate comparison of variable domains. In this system, conserved amino acids (such as Cys23, Trp41, Cys104, Phe/Trp118, and a hydrophobic residue at position 89) always have the same position. Additionally, a standardized delimitation of the framework regions (FR1: positions 1 to 26; FR2: 39 to 55; FR3: 66 to 104; and FR4: 118 to 129) and of the CDR (CDR1: 27 to 38, CDR2: 56 to 65; and CDR3: 105 to 117) is provided.

The CDR/loops are referred to herein according to the Kabat scheme for all CDR. The CDR of the antibodies of the present invention are referred to herein as CDR L1, L2, L3 for CDR in the light chain, and CDR H1, H2, H3 for CDR in the heavy chain.

An “antibody fragment” as referred to herein may include any suitable antigen-binding antibody fragment known in the art. The antibody fragment may be a naturally-occurring antibody fragment or may be obtained by manipulation of a naturally-occurring antibody or by using recombinant methods. For example, an antibody fragment may include, but is not limited to: a single domain antibody (sdAb), a Fv, single-chain Fv (scFv; a molecule consisting of VL and VH connected with a peptide linker), Fab, F(ab′)2, and multivalent presentations of any of these. Antibody fragments such as those just described may require linker sequences, disulfide bonds, or other type of covalent bond to link different portions of the fragments; those of skill in the art will be familiar with various approaches. The terms “antibody” and “antibody fragments” are used herein interchangeably, unless stated otherwise.

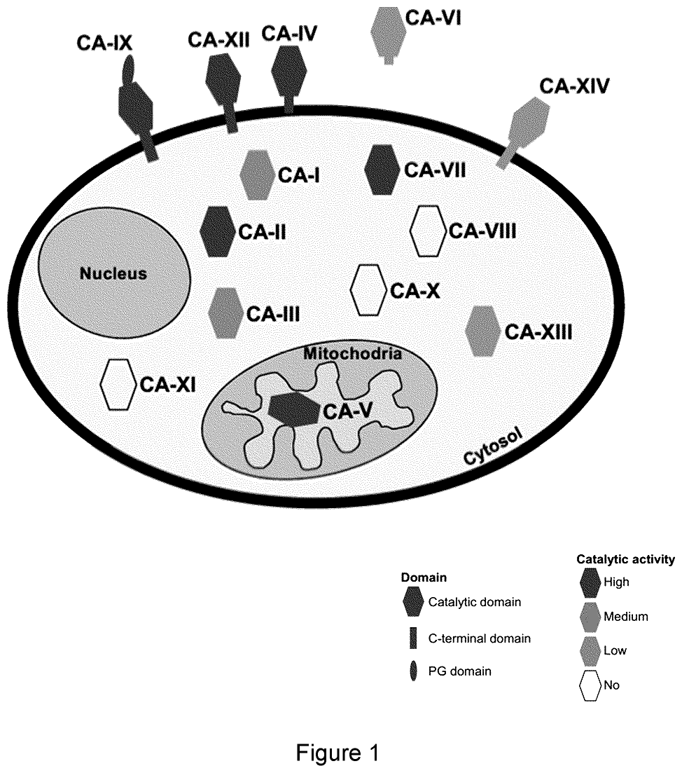

The antibody or fragment thereof of the present invention specifically binds to the extracellular domain of human (h)Carbonic Anhydrase (CA) IX (Genbank Accession no. NC_000009.12). CA-IX is a metalloenzyme that catalyzes the reversible hydration of carbon dioxide to bicarbonate and protons (FIG. 1). CA-IX is a transmembrane protein with an extracellular catalytic site and an NH₂-terminal proteoglycan (PG)-like domain. An antibody and a fragment thereof “specifically binds” CA-IX if it binds CA-IX with an equilibrium dissociation constant (KD, i.e., a ratio of Kd/Ka, Kd and Ka are the dissociation rate and the association rate, respectively) less than 10⁻⁵ M (e.g., less than 10⁻⁶ M, 10⁻⁷ M, 10⁻⁸ M, 10⁻⁹ M, 10⁻¹⁰ M, 10⁻¹¹ M, 10⁻¹² M, or 10⁻¹³ M), while not significantly binding other components present in a test sample (e.g., with a KD that is at least 10 times, such as 50 times or 100 times, more than KD for binding CA-IX). Affinities of an antibody and a fragment thereof disclosed herein, and CA-IX can be readily determined using the method described in Example 5 of the present disclosure.

The antibody or fragment thereof as described herein should exhibit a high degree of internalization. Without wishing to be bound by theory, the antibodies or fragments thereof presently described bind to the extracellular domain of CA-IX. The antibodies or fragments thereof are then internalized by the cell and delivered into subcellular organelles, including endosomes and lysosomes. The antibody or fragment thereof as described herein may also reduce cell viability. Antibody internalization may be measured by any appropriate methods known in the art, including antibody internalization assays offered by Life Technologies, Zap Antibody Internalization Kit by Advanced targeting Systems, and/or quantitative assessment described in Liao-Chan et al., 2015.

The terms “antibody” and “antibody fragment” (“fragment thereof”) are as defined above. As previously stated, the antibody or fragment thereof may be from any source, human, mouse, or other; may be any isotype, including IgA, IgD, IgE, IgG, and IgM; and may be any type of fragment, including but not limited to Fv, scFv, Fab, and F(ab′)₂.

### Antibody Sequences

The CDR regions of mAbs 4A2 and 9B6 were sequenced and yielded the following amino acid sequence for 4A2:

Light chain (Leader sequence-FR1-CDR1-FR2-CDR2-
FR3-CDR3-FR4):
(SEQ ID No. 6)
MDFQVQIFSFLLISASVILSRGQIVLTQSPAVMSAFPGEKVTMTCSASSS
VGYMHWYQQKAGSSPRLLIYDTSNLSSGVPARCSGSGSGTSYSLTISRME
AEDAATYYCQQWRSYPPTFGGGTKLEIK;
and
Heavy chain (Leader sequence-FR1-CDR1-FR2-CDR2-
FR3-CDR3-FR4):
(SEQ ID NO. 10)
MYLGLNCVFIVFLLKGVQSEVKLEESGGGLVQPGRSMKLSCVASGFTFSY
YWMDWVRQSPEKGLEWVAEIRLKSDNYATHYAESVKGRFTISRDDSKSSV
YLQMNNLRAEDTGIYYCTRAPHYYGYFDYWGQGTTLTVSS

whereas 9B6 antibody yielded amino acid sequences:

Light chain (Leader sequence-FR1-CDR1-FR2-CDR2-
FR3-CDR3-FR4:
(SEQ ID NO. 14)
METDTILLWVLLLWVPGSTGDIVLTQSPSSLAVSLGQRATISCKASQSVD
YDGNSYMNWFQQKPGQPPKLLIYEASSLESGIPARISGSGSGTDFTLNIH
PVEEEDAATYYCQQSYEGPYTFGGGTKLEIK;
and
Heavy chain (Leader sequence-FR1-CDR1-FR2-CDR2-
FR3-CDR3-FR4):
(SEQ IDS NO. 18)
MGWSCLILFLVAAATGVHSQVQLQQPGAELVKPGASVKLSCKASGYIFTT
KWINWVKQRPGQGLEWIGNIYPGSSNTYYNEKFKNKATLTVDKSSNTAHL
QLSSLTSEDSAVYYCARGIANWGQGTPVTVSA.

The sequences were analyzed for a consensus binding sequence by analyzing the CDR 1-3 regions (underlined) of the Variable Heavy (VH) and Variable Light (VL) chains. The results of this analysis indicate that the CDR regions of the VL regions differ significantly, whereas the VH regions of these mAbs show 82% homology.

| Alignment V_{L} CDR1-3 | | |
|---|---|---|
| 4A2 | SASSSV----- GYMHDTSNLSSQQWRSYPPT | (SEQ ID NO. 42) |
| 9B6 | KASQSVDYDGNSYMNEASSLESQQSYEGPYT | (SEQ ID NO. 43) |
| | .**.** .**:::*.*.*** . * * | |
| Alignment V_{H} CDR1-3 | | |
| 4A2 | GFIFSYYWMDEIRLKSDNYATHYAESVKGAPHYYGYFDY | (SEQ ID NO. 44) |
| 9B6 | GYIFTTKWINEIRLKSDNYATHYAESVKGAPHYYGYFDY | (SEQ ID NO. 45) |
| | *: *: *::***************************** | |

From this sequencing data, the following consensus sequence was determined for the CDR1-3 regions of both antibodies:

Light chain:
(SEQ ID NO. 30)

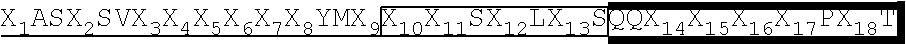

wherein
X₁ is S or K, X₂ is S or Q, X₃ is D or no amino acid, X₄ is Y or no amino acid,
X₅ is D or no amino acid, X₆ is G or no amino acid, X₇ is N or no amino acid, X₈ is G
or S, X₉ is H or N, X₁₀ is D or E, X₁₁ is T or A, X₁₂ is N or S, X₁₃ is S or E, X₁₄ is W or S,
X₁₅ is R or Y, X₁₆ S or E, X₁₇ Y or G and PX₁₈ is P or Y;
and
Heavy chain:
(SEQ ID NO. 31)

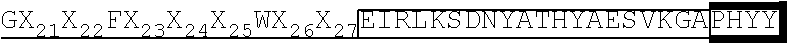

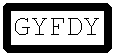

wherein
X₂₁ is F or Y, X₂₂ is T or I, X₂₃ is S or T, X₂₄ is Y or T, X₂₅ is Y or K, X₂₆
is M or I, X₂₇ is D or N.

Each CDR is represented above by a different underline or box. Hence, one consensus sequence is established for each CDR of these antibodies, one can surmise that antibodies comprising the consensus CDR will bind and/or inhibit human CA-IX.

In accordance with a further embodiment, there is provided an antibody or fragment thereof which comprises a light chain comprising:
a complementarity determining region (CDR) L1 comprising the sequence X₁ASX₂SVX₃X₄X₅X₆X₇X₈YMX₉ wherein X₁ is S or K, X₂ is S or Q, X₃ is D or no amino acid, X₄ is Y or no amino acid, X₅ is D or no amino acid, X₆ is G or no amino acid, X₇ is N or no amino acid, X₈ is G or S, Xg is H or N . . . (SEQ ID NO.32);
a CDR L2 comprising the sequence X₁₀X₁₁SX₁₂LX₁₃S wherein X₁₀ is D or E, Xu is T or A, X₁₂ is N or S, X₁₃ is S or E (SEQ ID NO.33); and
a CDR L3 comprising the sequence QQX₁₄X₁₅X₁₆X₁₇PX₁₈T wherein X₁₄ is W or S, X₁₅ is R or Y, X₁₆ S or E, X₁₇ Y or G and PX₁₈ is P or Y (SEQ ID NO.34);
or a sequence substantially identical thereto;

wherein the antibody or fragment is specific for CA-IX.

In accordance with an alternative embodiment, there is provided an antibody or fragment thereof which comprises a heavy chain comprising:
a complementarity determining region (CDR) H1 comprising a peptide defined by sequence: GX₂₁X₂₂FX₂₃X₂₄X₂₅WX₂₆X₂₇ wherein X₂₁ is F or Y, X₂₂ is T or I, X₂₃ is S or T, X₂₄ is Y or T, X₂₅ is Y or K, X₂₆ is M or I, X₂₇ is D or N (SEQ ID NO.35);
a CDR H2 comprising a peptide defined by sequence: EIRLKSDNYATHY AESVKGA (SEQ ID NO.36); and
a CDR H3 comprising a peptide defined by sequence: PHYYGYFDY (SEQ ID NO.37);
or a sequence substantially identical thereto;

wherein the antibody or fragment is specific for CA-IX.

In a particular embodiment, the antibody or fragment comprises:
a light chain comprising the CDRs L1-3 as defined herein; and
a heavy chain comprising the CDRs H1-3 as defined herein;
or a sequence substantially identical thereto.

More particularly, the antibody or fragment comprises a CDR L1 defined as:

(SEQ ID NO: 6)
SASSSVGYMH
or
(SEQ ID NO: 15)
KASQSVDYDGNSYMN
and/or
a CDR L2
defined as:
(SEQ ID NO: 7)
DTSNLSS
or
(SEQ ID NO: 16)
EASSLES;
and/or
a CDR L3
defined as:
SEQ ID NO: 8)
QQWRSYPPT
or
(SEQ ID NO: 17)
QQSYEGPYT

Still, particularly, the antibody or fragment comprises a CDR H1 defined as:

(SEQ ID NO: 11)
GFTFSYYWMD
or
(SEQ ID NO: 19)
GYIFTTKWIN;
and/or
a CDR H2
defined as:
(SEQ ID NO: 12)
EIRLKSDNYATHYAESVKG
or
(SEQ ID NO: 20)
NIYPGSSNTYYNEKFKN;
and/or
a CDR H3
defined as:
(SEQ ID NO: 13)
APHYYGYFDY
or
(SEQ ID NO: 21)
GIAN.

In accordance with a particular embodiment, there is provided an antibody designated as 4A2 that comprises a peptide sequence: comprising CDR L1-3 sequences SEQ ID NO.6, 7 & 8; and comprising CDR H1-3 sequences SEQ ID NO. 11, 12 & 13. More particularly the antibody 4A2 comprises a sequence defined by SEQ ID NO.6 and SEQ ID NO.10; or a sequence comprising SEQ ID NO. 22 and 23.

In accordance with an alternative embodiment, there is also provided an antibody designated as 9B6 comprises a peptide sequence: comprising CDR L1-3 sequences SEQ ID NO.15, 16 & 17; and comprising CDR H1-3 sequences SEQ ID NO. 19, 20 & 21. More particularly, the antibody 9B6 comprises a sequence defined by SEQ ID NO.14 and SEQ ID NO.18; or a sequence comprising SEQ ID NO. 22 and 23.

According to particular embodiments, the antibody or fragment is a full-length IgG, Fv, scFv, Fab, or F(ab′)₂. Alternatively, the antibody or fragment comprises framework regions from IgA, IgD, IgE, IgG, or IgM. More particularly, the antibody or fragment thereof is chimeric and may comprise a constant domain from human IgG2 and/or possibly with human kappa-1 light chain and human IgG2 heavy chain constant domains.

According to particular embodiment, there is provided a composition comprising one or more than one antibody or fragment as defined herein, in admixture with a pharmaceutically acceptable carrier, diluent, or excipient.

A substantially identical sequence may comprise one or more conservative amino acid mutations. It is known in the art that one or more conservative amino acid mutations to a reference sequence may yield a mutant peptide with no substantial change in physiological, chemical, physicochemical or functional properties compared to the reference sequence; in such a case, the reference and mutant sequences would be considered “substantially identical” polypeptides. A conservative amino acid substitution is defined herein as the substitution of an amino acid residue for another amino acid residue with similar chemical properties (e.g. size, charge, or polarity). These conservative amino acid mutations may be made to the framework regions of the antibody or fragment thereof while maintaining the CDR sequences listed above and the overall structure of the antibody or fragment; thus, the specificity and binding of the antibody are maintained.

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gin or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine ale or I), phenylalanine (Phe or F), valine (Val or V), leucine (Leu or L), tryptophan (Trp or VV), methionine (Met or M), alanine (Ala or A), and glycine (Gly or G). “Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), or any other appropriate software that is known in the art.

The substantially identical sequences of the present invention may be at least 90% identical; in another example, the substantially identical sequences may be at least 90, 91, 92, 93, 94, 95, 96, 97, 98, 99, or 100% identical, or any percentage there between, at the amino acid level to sequences described herein. Importantly, the substantially identical sequences retain the activity and specificity of the reference sequence. In a non-limiting embodiment, the difference in sequence identity may be due to conservative amino acid mutation(s). In a non-limiting example, the present invention may be directed to an antibody or fragment thereof comprising a sequence at least 95%, 98% or 99% identical to that of the antibodies described herein.

The present invention further encompasses an antibody or fragment thereof that is chimeric (or chimerized), veneered, or humanized. The antibody or fragment thereof may be chimeric, in that the antibody or fragment thereof is a combination of protein sequences originating from more than one species. As is known to those of skill in the art, a chimeric antibody is produced by combining genetic material from a nonhuman source (for example but not limited to a mouse) with genetic material from a human. For example, and without wishing to be limiting, human constant domains can be fused to mouse VH and VL sequences (see Gonzales et al., 2005). Veneering, also referred to in the art as “variable region resurfacing”, of antibodies involves replacing solvent-exposed residues in the framework region of the native antibody or fragment thereof with the amino acid residues in their human counterpart (Padlan, 1991; Gonzales et al., 2005); thus, buried non-humanized residues, which may be important for CDR conformation, are preserved while the potential for immunological reaction against solvent exposed regions is minimized. Humanization of an antibody or antibody fragment comprises replacing an amino acid in the sequence with its human counterpart, as found in the human consensus sequence, without loss of antigen-binding ability or specificity; this approach reduces immunogenicity of the antibody or fragment thereof when introduced into human subjects. In this process, one or more than one of the CDR defined herein may be fused or grafted to a human variable region (VH, or VL), to other human antibody (IgA, IgD, IgE, IgG, and IgM), to human antibody fragment framework regions (Fv, scFv, Fab), or to human proteins of similar size and nature onto which CDR can be grafted (Nicaise et al., 2004). In such a case, the conformation of said one or more than one hypervariable loop is likely preserved, and the affinity and specificity of the sdAb for its target (i.e., Axl) is likely minimally affected. As is known by those of skill in the art, it may be necessary to incorporate certain native amino acid residues into the human framework in order to retain binding and specificity. Humanization by CDR grafting is known in the art (for example, see Tsurushita et al., 2005; Jones et al., 1986; Tempest et al., 1991; Riechmann et al., 1988; Queen et al., 1989; reviewed in Gonzales et al., 2005—see also references cited therein), and thus persons of skill would be amply familiar with methods of preparing such humanized antibody or fragments thereof.

The present invention thus provides an isolated or purified antibody or fragment thereof specific for CA-IX may be a chimeric antibody comprising the variable domain as defined above linked to human IgG1 constant domains. For example, and without wishing to be limiting in any manner, the human IgG1 constant domains may comprise a human kappa 1 light chain constant domain and human IgG1 heavy chain constant domains.

The antibody or fragment thereof of the present invention may also comprise additional sequences to aid in expression, detection or purification of a recombinant antibody or fragment thereof. Any such sequences or tags known to those of skill in the art may be used. For example, and without wishing to be limiting, the antibody or fragment thereof may comprise a targeting or signal sequence (for example, but not limited to ompA), a detection/purification tag (for example, but not limited to c-Myc, His₅, His₆, or His₈G), or a combination thereof.

The antibody or fragment thereof of the present invention may also be in a multivalent display format, also referred to herein as multivalent presentation. Multimerization may be achieved by any suitable method known in the art. For example, and without wishing to be limiting in any manner, multimerization may be achieved using self-assembly molecules such as those described in Zhang et al (2004a; 2004b) and WO2003/046560. The described method produces pentabodies by expressing a fusion protein comprising the antibody or fragment thereof of the present invention and the pentamerization domain of the B-subunit of an AB5 toxin family (Merritt & Hoi, 1995); the pentamerization domain assembles into a pentamer. A multimer may also be formed using the multimerization domains described by Zhu et al. (2010); this form, referred to herein as a “combody” form, is a fusion of the antibody or fragment of the present invention with a coiled-coil peptide resulting in a multimeric molecule (Zhu et al., 2010). Other forms of multivalent display are also encompassed by the present invention. For example, and without wishing to be limiting, the antibody or fragment thereof may be presented as a dimer, a trimer, or any other suitable oligomer. This may be achieved by methods known in the art, for example direct linking connection (Nielson et al, 2000), c-jun/Fos interaction (de Kruif & Logtenberg, 1996), “knob into holes” interaction (Ridgway et al., 1996).

Each subunit of the multimers described above may comprise the same or different antibodies or fragments thereof of the present invention, which may have the same or different specificity. Additionally, the multimerization domains may be linked to the antibody or antibody fragment using a linker, as required; such a linker should be of sufficient length and appropriate composition to provide flexible attachment of the two molecules but should not hamper the antigen-binding properties of the antibody. For example, and without wishing to be limiting in any manner, the antibody or fragments thereof may be presented in a bi-specific antibody.

### Antigen-Binding Fragment

An antigen-binding fragment that specifically binds to a 6 to 12 amino acid peptide comprised within the epitope STAFARVDEALGR (SEQ ID NO. 5).

The antigen-binding fragment of claim 47, that specifically binds to a peptide selected from the group consisting: DEALGR (SEQ ID NO. 3) and STAFARVDE (SEQ ID NO. 4).

### Nucleic Acid

The present invention also encompasses nucleic acid sequences encoding the antibody or fragment as described herein. Particularly, nucleic acid comprises a sequence selected from the group consisting of: SEQ ID NO. 38, 39, 40 and 41.

Given the degeneracy of the genetic code, a number of nucleotide sequences would have the effect of encoding the desired polypeptide, as would be readily understood by a skilled artisan. The nucleic acid sequence may be codon-optimized for expression in various micro-organisms.

The present invention also encompasses vectors comprising the nucleic acids as just described. Furthermore, the invention encompasses cells comprising the nucleic acid and/or vector as described.

### Peptide Epitope of the Two Antibodies

The extracellular domain (ECD) of human CA-IX is defined by SEQ ID NO.1 (Signal peptide—hCA-W-His tag):

MAPLCPSPWLPLLIPAPAPGLTVQLLLSLLLLVPVHPQRLPRMQEDSPLG
GGSSGEDDPLGEEDLPSEEDSPREEDPPGEEDLPGEEDLPGEEDLPEVKP
KSEEEGSLKLEDLPTVEAPGDPQEPQNNAHRDKEGDDQSHWRYGGDPPWP
RVSPACAGRFQSPVDIRPQLAAFCPALRPLELLGFQLPPLPELRLRNNGH
SVQLTLPPGLEMALGPGREYRALQLHLHWGAAGRPGSEHTVEGHRFPAEI
HVVHLSTAFARV [symbol] PGGLAVLAAFLEEGPEENSAYEQLLSRLEEIA
EEGSETQVPGLDISALLPSDFSRYFQYEGSLTTPPCAQGVIWTVFNQTVM
LSAKQLHTLSDTLWGPGDSRLQLNFRATQPLNGRVIEASFPAGVDSSPRA
AEPVQLNSCLAAGDGSHHHHHHHHHHG and was used as the
antigen for producing the present antibodies.

The minimal epitopes of the antibodies were determined by epitope mapping using Yeast Surface Display. The minimal epitope for 4A2 was determined to be DEALGR (SEQ ID NO.3; corresponding to aa 263-268 of hCA-IX); and that of 9B6 was determined to be STAFARVDE (SEQ ID NO.4; corresponding to aa 256-264 of CA-IX). Thus, considering the 2 amino acids overlap between SEQ ID NO.3 and SEQ ID NO.4, one can surmise that the sequence epitope: STAFARVDEALGR (SEQ ID NO.5), when used as a peptide antigen for immunizing mice, could induce the production of antibodies directed against the catalytic activity of hCA-IX.

It is therefore another aspect of the present invention to provide a “universal epitope” for inducing and producing anti-CA-IX antibodies that bind to the catalytic site of CA-IX, wherein this universal epitope is as a peptide of 6 to 60 amino acids comprising the peptide sequence defined as RVDEAL (SEQ ID NO.46), more particularly, the peptide antigen comprises: STAFARVDEALGR (SEQ ID NO.5).

### Composition

The present invention also encompasses a composition comprising one or more than one antibody or fragment thereof as described herein. The composition may comprise a single antibody or fragment as described above or may be a mixture of antibodies or fragments. Furthermore, in a composition comprising a mixture of antibodies or fragments of the present invention, the antibodies may have the same specificity, or may differ in their specificities; for example, and without wishing to be limiting in any manner, the composition may comprise antibodies or fragments thereof specific to CA-IX (same or different epitope).

In accordance with a particular embodiment, there is provided a composition comprising the antibody 4A2 as defined herein, in combination with the antibody 9B6 as defined herein. More particularly, since these antibodies are directed toward different peptide epitope of the catalytic domain of CA-IX, the combination of these two mAbs has shown to be synergistic, both with respect to the full mAb or their respective Fab fragments.

The composition may also comprise a pharmaceutically acceptable diluent, excipient, or carrier. The diluent, excipient, or carrier may be any suitable diluent, excipient, or carrier known in the art, and must be compatible with other ingredients in the composition, with the method of delivery of the composition, and is not deleterious to the recipient of the composition. The composition may be in any suitable form; for example, the composition may be provided in suspension form, powder form (for example, but limited to lyophilised or encapsulated), capsule or tablet form. For example, and without wishing to be limiting, when the composition is provided in suspension form, the carrier may comprise water, saline, a suitable buffer, or additives to improve solubility and/or stability; reconstitution to produce the suspension is made in a buffer at a suitable pH to ensure the viability of the antibody or fragment thereof. Dry powders may also include additives to improve stability and/or carriers to increase bulk/volume; for example, and without wishing to be limiting, the dry powder composition may comprise sucrose or trehalose. In a specific, non-limiting example, the composition may be so formulated as to deliver the antibody or fragment thereof to the gastrointestinal tract of the subject. Thus, the composition may comprise encapsulation, time release, or other suitable technologies for delivery of the antibody or fragment thereof. It would be within the competency of a person of skill in the art to prepare suitable compositions comprising the present compounds.

### Use of the Antibodies

In a particular embodiment, the antibody or fragment as defined herein, for use in the manufacture of a composition for the treatment or prevention of cancer in a subject. Particularly, the cancer is a hCA-IX-expressing cancer, such as, but not limited to, Clear Cell Renal Cell Carcinoma (CCRCC) and the subject is a human or an animal.

According to an alternative embodiment, there is provided the antibody or fragment as defined herein, for use in the treatment or prevention of cancer in a subject. Particularly, the cancer is a hCA-IX expressing-cancer, more particularly Clear Cell Renal Cell Carcinoma (CCRCC) and the subject is a human or an animal.

Alternatively, there is provided the use of the antibody or fragment as defined herein, as a companion diagnostic in the adjuvant treatment of cancer. Particularly, the cancer is a hCA-IX-expressing cancer, more particularly Clear Cell Renal Cell Carcinoma (CCRCC).

The antibody or fragment as described herein may be immobilized onto a surface or may be linked to a cargo molecule. The cargo molecule may be a detectable agent, a therapeutic agent, a drug, a peptide, an enzyme, a growth factor, a cytokine, a receptor trap, an antibody or fragment thereof (e.g., IgG, scFv, Fab, VHH, etc) a chemical compound, a carbohydrate moiety, DNA-based molecules (anti-sense oligonucleotide, microRNA, siRNA, plasmid), a cytotoxic agent, viral vector (adeno-, lenti-, retro-), one or more liposomes or nanocarriers loaded with any of the previously recited types of cargo molecules, or one or more nanoparticle, nanowire, nanotube, or quantum dots. In a specific, non-limiting example, the cargo molecule is a cytotoxic agent.

### Method of Treatment

In accordance with a particular aspect, there is provided a method of preventing or treating cancer in a subject, comprising administering a pharmaceutically acceptable dose of an antibody or fragment as defined herein to the subject. Particularly, the cancer is a hCA-IX-expressing cancer, such as, but not limited to, Clear Cell Renal Cell Carcinoma (CCRCC) and the subject is a human or an animal.

### Other Methods

There is also provided a method of detecting expression of Carbonic anhydrase-IX (CA-IX) in a biological sample, comprising the steps of: a) contacting the biological sample, with the antibody or fragment as defined herein, linked to a detectable agent; and b) detecting the detectable agent linked to the antibody or fragment thereof bound to CA-IX in the biological sample. Particularly, the steps are carried in situ in a subject, and the biological sample is from: blood or an organ (such as a kidney) and is a human or an animal. Alternatively, the steps are carried in vitro, and the biological sample is from: blood, serum, urine or a biopsy tissue (such as a tumor tissue and/or a kidney tissue).

According to a particular embodiment of the method of detection, the step of detecting step b) is performed using optical imaging, immunohistochemistry, molecular diagnostic imaging, ELISA, or other suitable method.

In another particular embodiment, there is provided a method of inhibiting Carbonic anhydrase-IX (CA-IX) enzymatic activity in a cell, comprising contacting the cell with the antibody or fragment as defined herein, optionally linked to a chemotherapeutic drug.

Thus, the present invention further provides an in vitro method of detecting CA-IX, comprising contacting a tissue sample with one or more than one isolated or purified antibody or fragment thereof of the present invention linked to a detectable agent. The CA-IX-antibody complex can then be detected using detection and/or imaging technologies known in the art. The tissue sample in the method as just described may be any suitable tissue sample, for example but not limited to a serum sample, a vascular tissue sample, or a tumour tissue sample; the tissue sample may be from a human or animal subject. The step of contacting is done under suitable conditions, known to those skilled in the art, for formation of a complex between the antibody or fragment thereof and CA-IX. The step of detecting may be accomplished by any suitable method known in the art, for example, but not limited to optical imaging, immunohistochemistry, molecular diagnostic imaging, ELISA, or other suitable method. For example, and without wishing to be limiting in any manner, the isolated or purified antibody or fragment thereof linked to a detectable agent may be used in immunoassays (IA) including, but not limited to enzyme IA (EIA), ELISA, “rapid antigen capture”, “rapid chromatographic IA”, and “rapid EIA”. (For example, see Planche et al., 2008; Sloan et al., 2008; Russmann et al., 2007; Musher et al., 2007; Turgeon et al., 2003; Fenner et al., 2008). In a specific, non-limiting embodiment, the in vitro method is for detection of CA-IX in circulating cells and the tissue sample is a serum sample.

The present invention further provides an in vivo method of detecting CA-IX expression in a subject, comprising: a) administering one or more than one isolated or purified antibody or fragment thereof as described herein linked to a detectable agent to the subject; and b) detecting the detectable agent linked to the antibody or fragment thereof bound to CA-IX. In the method described just described, the step of detecting (step b)) is performed using PET, SPECT, fluorescence imaging, or any other suitable method. The method as just described may be useful in detecting the expression of CA-IX in tissues, for example but not limited to tumor tissues.

The in vivo detection step in the methods described above may be whole body imaging for diagnostic purposes or local imaging at specific sites, such as but not limited to sites of solid tumor growth, in a quantitative manner to assess the progression of disease or host response to a treatment regimen. The detection step in the methods as described above may be immunohistochemistry, or a non-invasive (molecular) diagnostic imaging technology.

The present invention will be further illustrated in the following examples. However, it is to be understood that these examples are for illustrative purposes only and should not be used to limit the scope of the present invention in any manner.

The following examples are put forth to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES—MONOCLONAL ANTIBODIES AGAINST CA-IX

A set of fifty-one (51) monoclonal antibodies (mAbs) was generated by immunizing A/J mice with extracellular portion of the rhCA-IX ECD protein (SEQ ID NO.1). This set of mAbs was characterized using various biophysical and functional assays (both cell-based and non-cell based). Two mAbs (designated 4A2 and 9B6) were identified as hCA-IX enzyme inhibiting mAbs.

A total of 51 mAbs have been tested in a non-purified format for antigen recognition by Enzyme-Linked ImmunoSorbent Assay (ELISA), Reverse Phase Protein Array (RPPA), Surface Plasmon Resonance (SPR), and western blot analysis. MAbs were then purified (Protein A affinity) and retested for binding to the rhCA-IX ECD monomer and dimer (obtained by size exclusion chromatography, SEC) by SPR. In addition, mAbs were evaluated for: a) binding to cell surface expressed hCA-IX by flow cytometry (FC), and b) hCA-IX enzyme blocking activity.

### Example 1—Production of Hybridoma-Derived mAbs

rhCA-IX ECD Expression

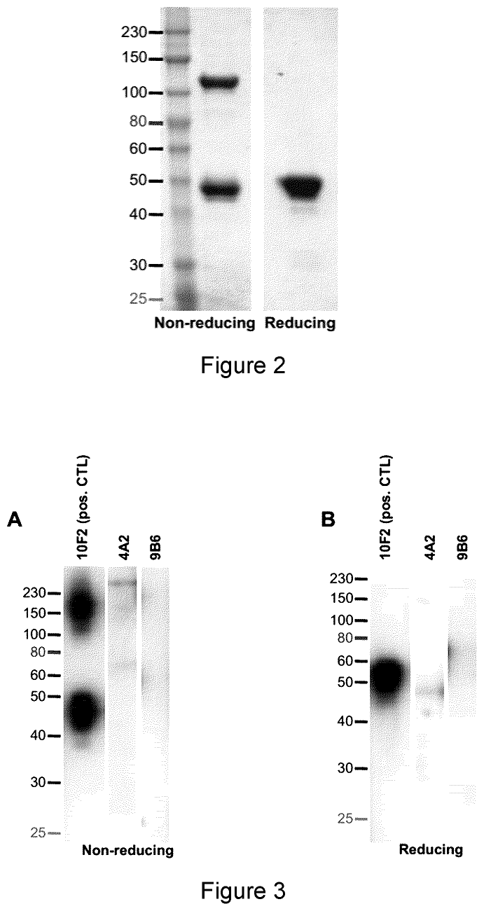

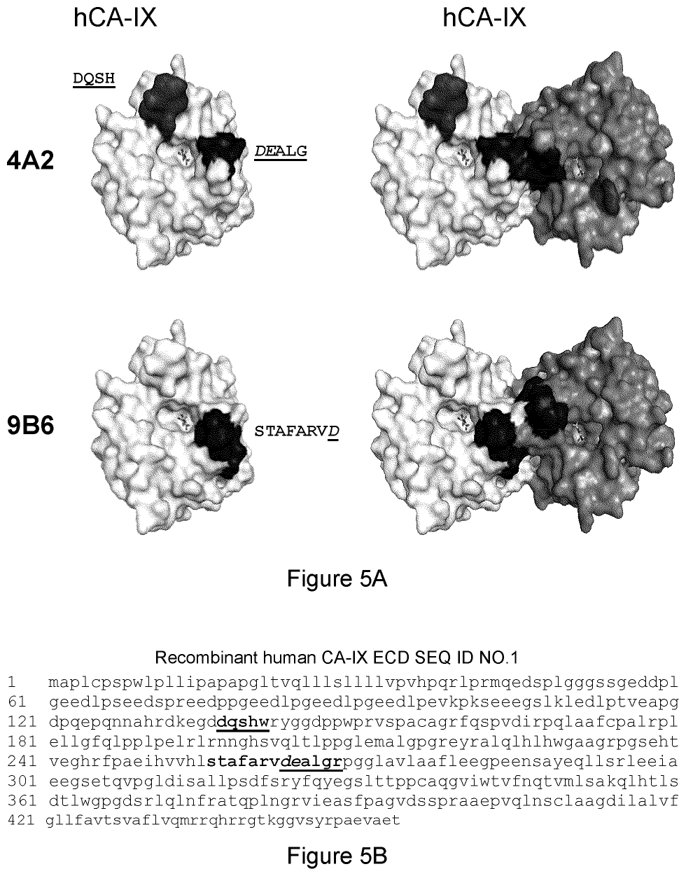

C-terminally His-tagged recombinant human (rh)CA-IX extracellular domain (ECD) (FIG. 5B), was expressed in CHO cells, purified by Ni-agarose and verified by Sodium Dodecyl Sulfate PolyAcrylamide Gel Electrophoresis (SDS-PAGE) under non-reducing and reducing conditions (FIG. 2). The protein was shown to be a 50/50 mixture of hCA-IX ECD monomer (˜48 kDa) and dimer (˜110 kDa).

### Antibody Generation and Characterization

The rhCA-IX monomer/dimer mixture was used to immunize A/J mice. After fusion of the harvested spleen cells, 51 CA-IX mAb producing hybridomas were identified, from which conditioned medium (CM) was collected and evaluated for binding to the rhCA-IX ECD protein by ELISA (Table 3).

TABLE 3
Evaluation of the CM collected from the mAb
producing hybridomas by ELISA (rhCA-IX ECD).

| Fusion | Clone | Species | Isotype | ELISA on protein |
|---|---|---|---|---|
| F117 | 4A2 | mouse | IgG2b | +++ |
| F101 | 9B6 | mouse | IgG2b | +++ |

### Example 2—Binding Epitope Characterization

### Western Blot & RPPA

Western blot analysis of the non-purified mAbs 4A2 or 9B6 indicated that these mAbs do not detect rhCA-IX ECD under these conditions (FIG. 3). The Reverse Phase Protein Array (RPPA; Table 4) showed: a) the specificity of these mAbs for rhCA-IX (i.e. no other proteins were detected in the protein mixture), and b) that both mAbs bind to both native and denatured rhCA-IX ECD. mAb 4A2 binds slightly better to the native protein, whereas mAb 9B6 seems to have a slightly increased preference for the denatured protein.

TABLE 4
RPPA results showing the hCA-IX ECD binding specificity of mAbs 4A2 and 9B6.

| | | Purified Antigen | | | | | | |
|---|---|---|---|---|---|---|---|---|
| | | | | Ratio | | | | |
| | | | | (A)/(B) | Antigen in protein | | | mAb |
| | | | | (Possibly | mixture | | mAb | concentration |
| | | Native | Denatured | epitope | Native | Denatured | binding | in hybridoma |
| Fusion | Clone | (A) | (B) | related) | (C) | (D) | specificity | CM (μg/ml) |
| F117 | 4A2 | 29.54 | 19.14 | 1.54 | 8.9 | 0.13 | rhCA-IX | 24.57 |
| | | | | | | | ECD | |
| F101 | 9B6 | 14.75 | 21.55 | 0.68 | 5.79 | 1.74 | rhCA-IX | 21.73 |
| | | | | | | | ECD | |

mAb & rhCA-IX ECD Purification

All mAbs were then Protein A purified (spin column), dialyzed twice and concentrated. The final concentration of the antibody solutions was determined by nano-drop (Table 5).

TABLE 5
Hybridoma 4A2 and 9B6 mAb concentration from 50 mL CHO
culture by nanodrop after Protein A purification

| | | Final | Final | Final |
|---|---|---|---|---|
| | | volume | concentration | yield |
| Fusion | Clone | (uL) | (μg/uL) | (μg) |
| F117 | 4A2 | 200 | 0.78 | 156 |
| F101 | 9B6 | 370 | 0.93 | 344 |

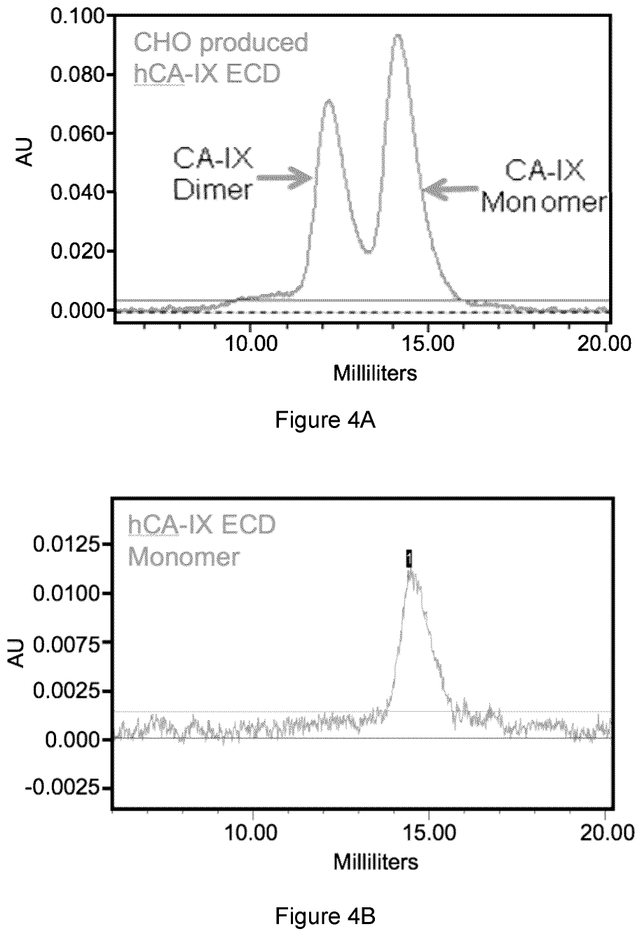

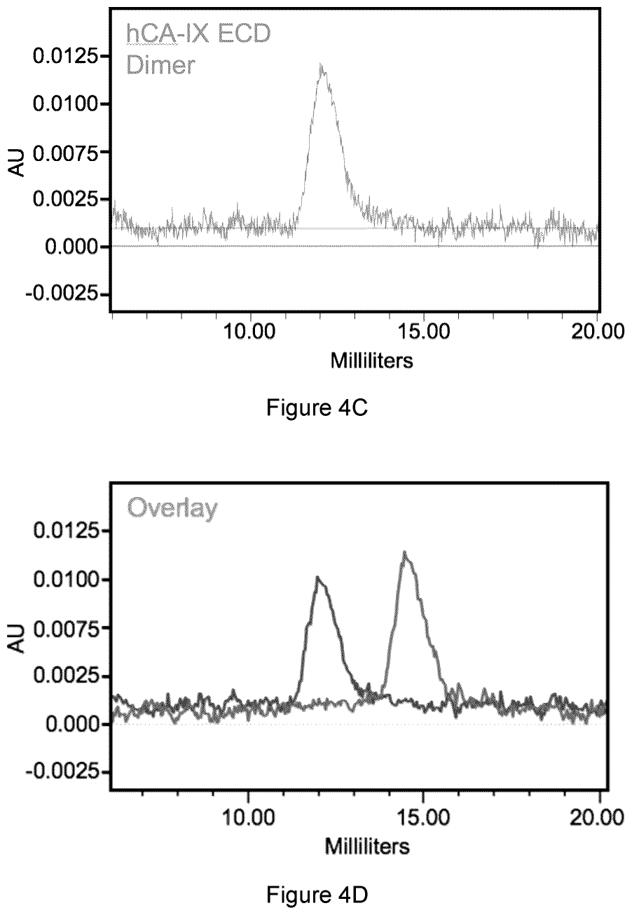

Since the mAbs were generated by immunizing the mice with the 50/50 mixture hCA-IX ECD monomer and dimer, we asked ourselves whether the generated mAbs would have a preference for binding the monomer or dimer. We thus performed a size exclusion (SEC) of the protein to separate the rhCA-IX monomers and dimers (FIG. 4A). Fractions with the highest protein concentration were selected (Table 6, bold font) and kept at 4° C. for several weeks. Prior to further Surface Plasmon Resonance (SPR) measurements, samples were reanalyzed by SEC (FIG. 4B-D). The results of this evaluation showed that both the monomer and dimer remain stable upon storage.

TABLE 6
OD280 of the 1 mL hCA-IX monomer and dimer containing
fractions collected from the G200 column

| | OD280 | |
|---|---|---|
| Fraction | (1 Abs = 1 | |
| number | mg/mL) | |
| F22 | 0.06 | Monomer |
| F21 | 0.12 | |
| F20 | 0.09 | |
| F19 | −0.06 | |
| F18 | 0.05 | Dimer |
| F17 | 0.11 | |
| F16 | 0.07 | |
| F15 | 0 | |

### SPR

SPR experiments were carried out by capturing the CA-IX mAbs from the CM with an anti-mouse Fc antibody immobilized on the chip surface, after which rhCA-IX EDC mixture, or rhCA-IX EDC monomer or dimer were flowed over the mAb surface. The results of these experiments (Table 7) show that mAb 4A2 has a slight preference for the rhCA-IX dimer over the monomer. No data could be obtained for mAb 9B6 when using the rhCA-IX ECD monomer+dimer mixture or the rhCA-IX ECD monomer alone. mAb 9B6 prefers to bind the rhCA-IX ECD dimer. The ka of this mAb is very similar to that of mAb 4A2, however its off-rate (kd) is extremely slow.

TABLE 7
Overview SPR results of purified hybridoma-derived mAbs 4A2 and 9B6 using hCA-IX ECD monomer and dimer preps.

| | | | rhCA-IX ECD Mixture | | | | | | | | | | |
|---|---|---|---|---|---|---|---|---|---|---|---|---|---|
| | | | (Monomer + Dimer) | | | | | | | | | | |
| | | | | | | | SPR | | | | | | |
| | | mAb | | | | | result | | | | | | |
| | | capture | | | | | (over- | rhCA-IX ECD Monomer | | | rhCA-IX ECD Dimer | | |
| Fusion | Clone | levels | ka | kd | KD | SPR | all) | ka | kd | KD | ka | kd | KD |
| F117 | 4A2 | 400 | 2.59E+05 | 2.29E−04 | 8.82E−10 | slow | ++ | 3.50E+05 | 3.43E−03 | 1.38E−09 | 2.82E+06 | 3.66E−05 | 1.60E−10 |
| | | | | | | off | | | | | | | |
| F101 | 9B6 | 400 | − | − | − | − | +/− | − | − | − | 1.33E+05 | 4.17E−18 | 3.13E−23 |

### Binding Epitope Characterization

PepScan: To gain insight into the binding epitope of the 4A2 and 9B6 mAbs, we used the PepScan technology (http://www.pepscan.com). This technology applies the CLIPS™ (Chemical Linkage of Peptides onto Scaffolds) method, which uses 15 amino acid long synthetic peptides (covering the entire hCA-IX protein), that are flanked by a cysteine in addition to a synthetic scaffold containing a benzyl-bromide group (Timmerman et al., 2009). The chemical linkage of the homocysteines to a solid surface offers a ‘linear’ and single ‘loop’ format of the peptides against which the mAbs were screened. Peptide sequences which, after mAb binding, generated a signal that exceeded the ‘mean+/−2× standard deviation’ were selected and aligned. Consensus sequences were then projected on the known 3D crystal structure of the monomer and dimer hCA-IX catalytic domain (FIG. 5A; Alterio et al., 2009), which thus allowed for the elimination of peptides that would, based on their position in the structure, not be accessible to the mAbs.

Using this approach, a plausible binding epitope for mAb 4A2 was determined to contain the ‘DQSHW’ (SEQ ID NO.2) or the ‘DEALGR’ (SEQ ID NO.3) sequence (FIG. 5B). For the 9B6 mAb the likely binding epitope was determined to contain the ‘STAFARVDE’ (SEQ ID NO.4) sequence. Further evaluation shows that the ‘STAFARVDE’ (SEQ ID NO.4) and ‘DEALGR’ (SEQ ID NO.3) sequence form a larger epitope (SEQ ID NO.5) that is close to the active center of hCA-IX and that would allow for these mAbs to: a) block CA-IX activity, and to do this in b) a non-competitive manner.

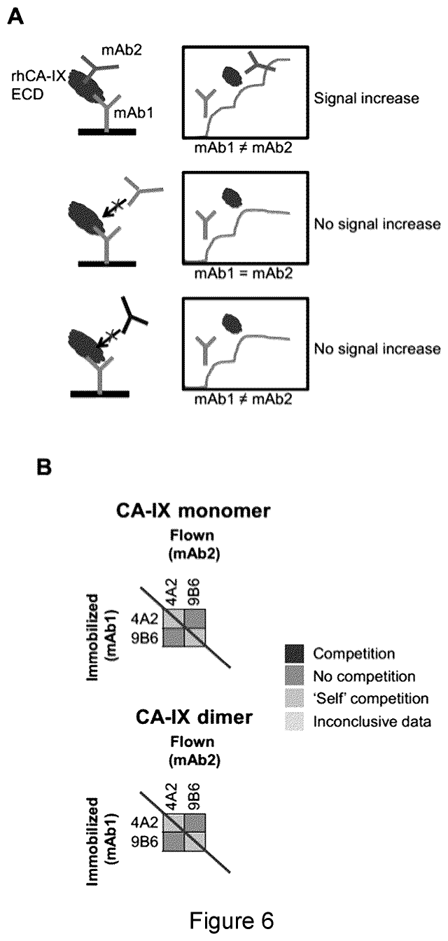

Epitope binding by SPR: To evaluate whether mAbs truly belong to the different classes, i.e. they do not bind to the same epitope region, SPR epitope binning experiments were carried out with the mAbs 4A2 and 9B6. For this mAbs were directly immobilized on the chips surface (‘mAb1’), after which either the rhCA-IX ECD monomer or dimer was flowed, followed by flowing the same mAbs (‘mAb2’). Immobilization of mAb1 will generate a signal, which will increase after the rhCA-IX ECD monomer or dimer binds and will increase again only when mAb2 flowed over this surface does not compete for binding to rhCA-IX ECD monomer with mAb1 (FIG. 6A). The generated data indicates that these mAbs do not compete for binding to either the rhCA-IX ECD monomer or dimer (FIG. 6B).

### Example 3—Functional Characterization

In Vitro rhCA-IX Enzyme Activity Inhibition

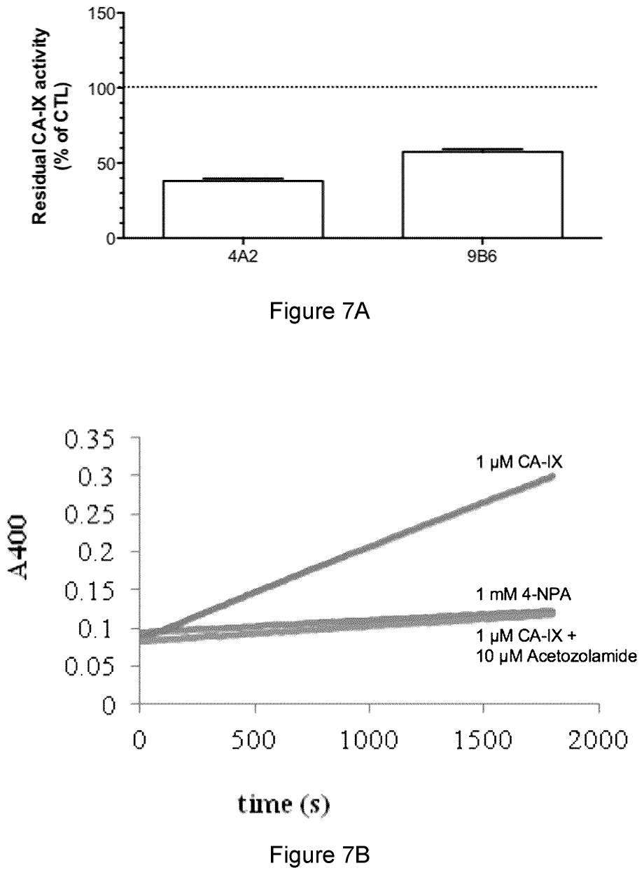

To determine whether these mAbs can inhibit the enzyme activity of the rhCA-IX ECD protein, we used an activity assay in which 4-Nitrophenyl acetate (4-NPA) is used as a substrate. Using a 1 μM rhCA-IX ECD (dimer), mAbs were then evaluated for their ability to inhibit rhCA-IX enzyme activity in a 4:1 molar mAb:rhCA-IX ECD ratio. Both mAbs showed significant enzyme inhibiting activity, with mAb 4A2 inhibiting rhCA-IX ECD enzyme activity with 61.94% and mAb 9B6 with 42.59%. The small molecule inhibitor acetazolamide was used as a positive control and blocked activity completely (FIG. 7, Table 8).

TABLE 8
Overview of the rhCA-IX enzyme inhibition data for hybridoma-derived mAbs
4A2 and 9B6 using the rhCA-IX ECD dimer (mAb:rhCA-IX ECD mixture = 4:1)

| | | | | % rhCA-IX | | % rhCA-IX | | |
|---|---|---|---|---|---|---|---|---|
| | | | | ECD | | ECD | | SEQ ID |
| Fusion | Clone | Data1 | Data2 | Activity | STDEV | Inhibition | Comments | NO. |
| F117 | 4A2 | 39.64 | 36.47 | 38.055 | 2.241528 | 61.945 | Inhibition | 6 & 10 |
| F101 | 9B6 | 59.25 | 55.57 | 59.25 | 2.602153 | 40.75 | Inhibition | 14 & 18 |

### Cell Line Verification & Flow Cytometry

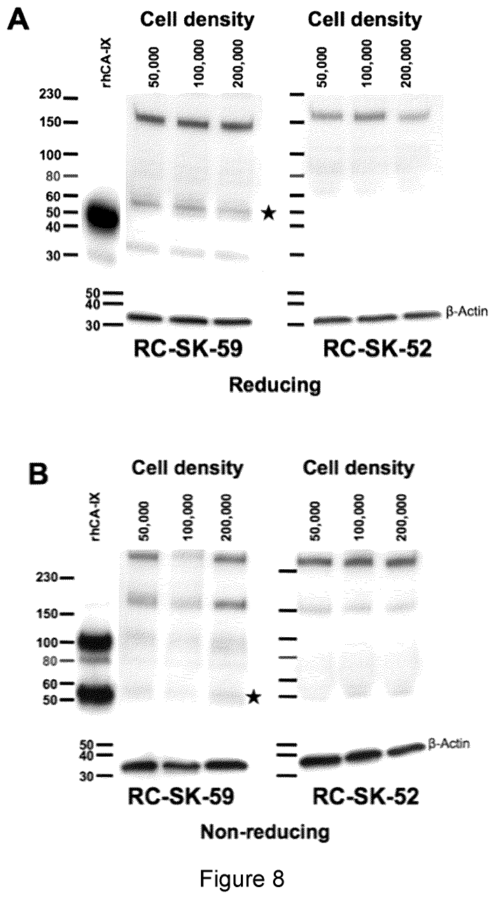

Western blot: To evaluate binding of the CA-IX mAbs to cell surface expressed CA-IX, we obtained the non-transfected human renal tumor SK-RC-59 and SK-RC-52 cell lines (a generous gift from DrG Ritter; Memorial Sloan Kettering Cancer Center, NY, USA), which have been shown to constitutively express either high (SK-RC-52) or low (SK-RC-59) hCA-IX levels. These cell lines have been used in the literature for the screening of scFv Abs for binding to hCA-IX (Xu et al., 2011). Western blot analysis of these cell lines under reducing and non-reducing conditions (FIG. 8) confirmed the expression of hCA-IX in the SK-RC-52 cell line (indicated with a star), whereas no hCA-IX could be detected in the SK-RC-59 cell line. Equal protein quantities were loaded (BCA protein assay) which was confirmed by an actin blot.

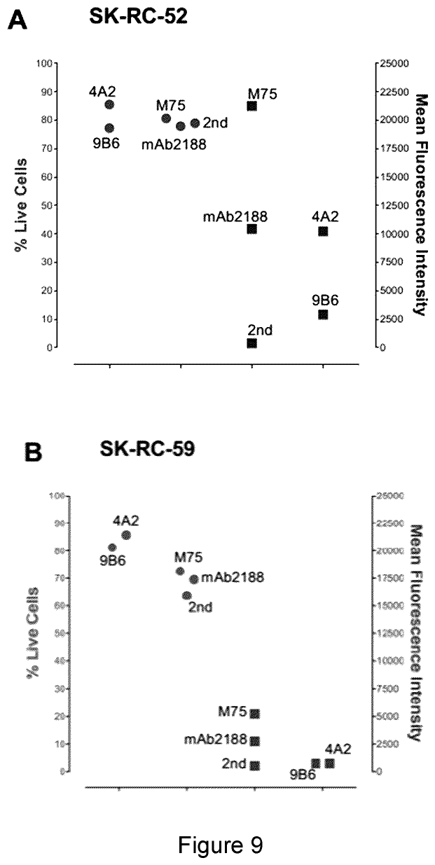

Flow cytometry: mAbs were evaluated in a flow cytometry experiment using the SK-RC-59 and SK-RC-52 cell lines. As controls a commercially available mAb (R&D, Clone #303123, Cat #MAB2188) and the M75 mAb (generously supplied by Dr E Oosterwijk, Radboud University Nijmegen, The Netherlands) were used. Experiments were plotted per cell line (FIG. 9) using a double Y-axis (left Y-axis, % live cells, closed squares; right Y-axis, mean fluorescence intensity of the live population, closed circles; X-axis, experimental groups). These results showed that mAb 4A2 binds slightly better to hCA-IX expressed by the SK-RC-52 cell line compared to 9B6, whereas virtually no binding was detected on the low hCA-IX expressing SK-RC-59 cells.

### Example 4—Experiments Using the Recombinantly-Expressed Chimeric mAbs

### Antibody Sequencing

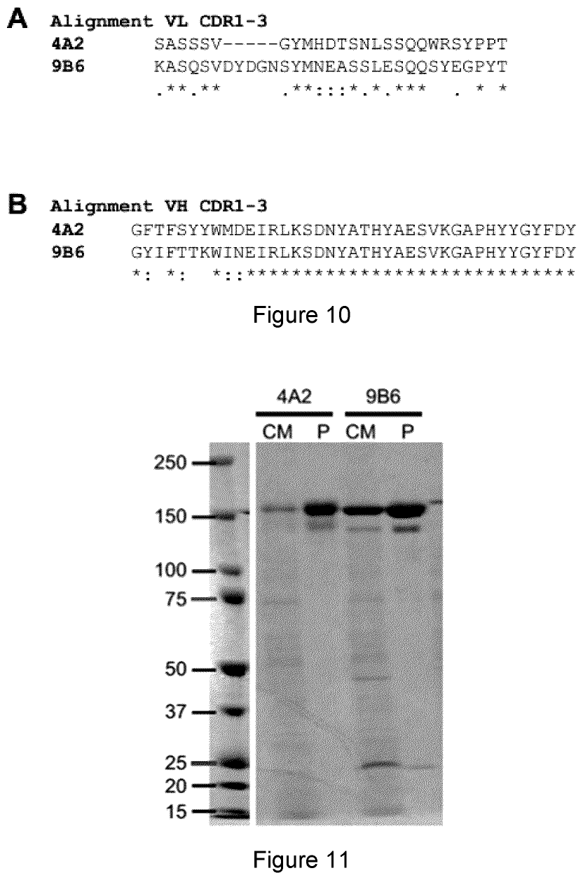

To facilitate large-scale mAb productions and consistency between these productions, we aimed to generate these mAbs recombinantly in CHO cells. For this the CDR regions of mAbs 4A2 and 9B6 were sequenced and analyzed for a consensus binding sequence by analyzing the CDR 1-3 regions of the Variable Heavy (VH) and Variable Light (VL) chains using MUSCLE 3.7 software (DeReeper et al., 2008; phylogeny.lirmm.fr/phylo_cgi/index.cgi). The results of this analysis (FIG. 10) indicate that the CDR regions of the VL regions differ significantly, whereas the VH region of these mAbs shows a 82% homology.

### Recombinant Antibody Production and Purification

The CDR regions of mAbs 4A2 and 9B6 were then cloned in a mouse IgG2b backbone into the pTT5 vector, thereby generating recombinant mAbs (mouse back bone, mouse CDR). MAb expression was validated through a 2 mL expression scout: CHO cells were transiently transfected with VL and VH containing constructs (1:1 ratio). Conditioned medium (CM) was harvested on day 7 and mAb expression levels were evaluated by SDS-PAGE (data not shown), after which a small-scale production (50 mL) of both the 4A2 and 9B6 mAbs was initiated by transiently transfecting CHO cells with the same construct ratio. Conditioned medium (CM) was harvested on day 7, chimeric mAbs were purified (ProtA), quantitated (Table 9), and evaluated by SDS-PAGE (FIG. 11). These data show that both mAbs are well expressed by the transiently transfected CHO cells.

TABLE 9
Recombinant mouse 4A2 and 9B6 mAb concentration from 50
mL CHO culture by nanodrop after Protein A purification

| | | | Volume | Concentration | Final yield | SEQ ID |
|---|---|---|---|---|---|---|
| Clone | Frame work | Isotype | (μL) | (μg/μL) | (μg/50 mL) | NO. |
| 4A2 | Mouse | IgG2b | −500 | 1.71 | 855 | 22 & 23 |
| 9B6 | Mouse | IgG2b | −500 | 3.053 | 1526.5 | 24 & 25 |

### Example 5—Biophysical Characterization

### SPR

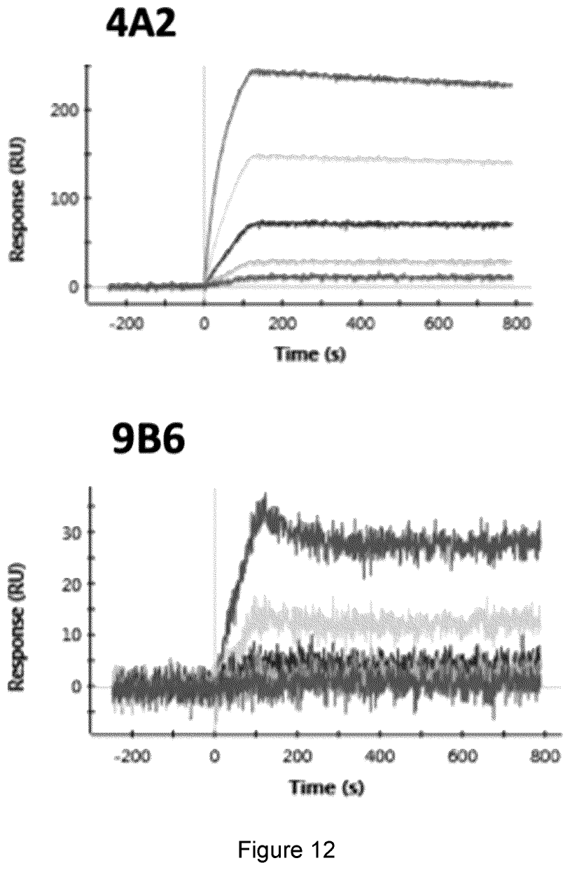

To confirm that these recombinantly expressed mAbs behave similarly to the hybridoma expressed mAbs SPR experiments were carried out by capturing the 4A2 and 9B6 mAbs with an anti-mouse Fc antibody immobilized on the chip surface after which rhCA-IX EDC dimer was flowed over the surface. Like the hybridoma-derived 4A2, the recombinant 4A2 has a very slow off-rate, whereas the kinetics of mAb 9B6 are poor in both cases (FIG. 12; see also Table 7).

These data confirmed that the binding characteristics of the recombinantly expressed 4A2 and 9B6 mAbs are similar to the original hybridoma expressed mAbs.

### Cross-Reactivity Determination by SPR

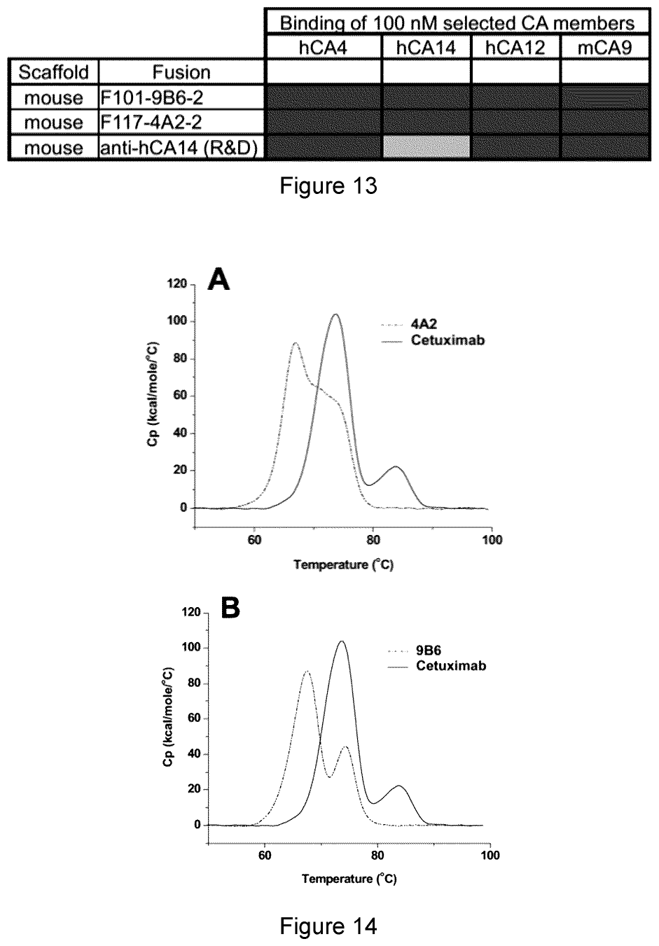

hCA-IX is one of several hCAs that have been identified to date (see FIG. 1). Although these hCAs differ in size expression in cells or tissue or secreted, hCA-IX displays a strong homology to especially hCA-XII. In contrast to hCA-IX the expression pattern of hCA-XII is much more widespread and not as cancer specific as hCA-IX. To evaluate whether the 4A2 and 9B6 mAbs are specific for hCA-IX, an SPR experiment was conducted in which the binding of these mAbs was evaluated using recombinant human (rh)CA-XII but also rhCA-IV and rhCA-XIV all of which are cell membrane associated hCAs with an extracellular active site. In addition, the cross reactivity of the 4A2 and 9B6 mAbs was tested for recombinant murine (rm)CA-IX; this cross reactivity would be an advantage in later in vivo studies using a syngeneic model system. The results indicate that the 4A2 and 9B6 mAbs are specific for the hCA-IX whereas no interaction was detected for rhCA-IV, rhCA-XII and rhCA-XIV, or rmCA-IX (FIG. 13).

### Differential Scanning Calorimetry (DSC) Evaluation

To determine the thermostability of the 4A2 and 9B6 mAbs, a DSC evaluation was performed. Briefly, the stability of the 4A2 and 9B6 mAbs was monitored over time at increasing temperatures between 25° C. and 100° C. (scan rate 90° C./hour). The therapeutic anti-EGFR Ab Cetuximab, which has been proven to be a very stable under these conditions, was used as control. The 4A2 and 9B6 mAbs display a similar thermostability however these mAbs are slightly less stable than Cetuximab (FIG. 14).

### Epitope Mapping by Yeast Surface Display (YSD)

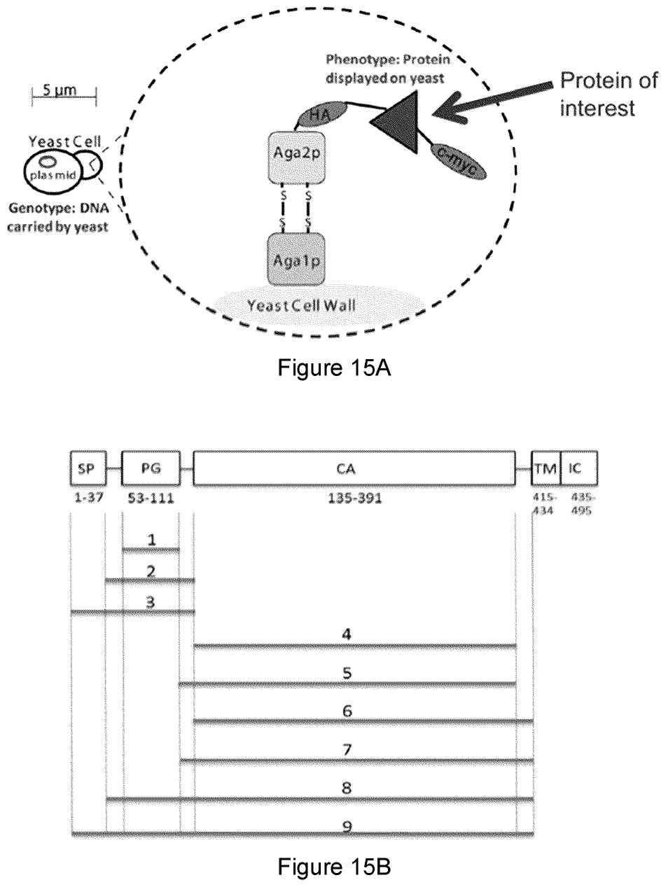

Yeast Surface Display (YSD; developed by the Wittrup lab) is used for presenting properly folded (ensured by the “quality-control” processes of the secretory pathway of the yeast) antigen fragments on the yeast cell surface through covalent linkage (FIG. 15A). We used this approach to: a) epitope map the amino acid sequence to which the chimeric mAbs 4A2 and 9B6 bind, and b) evaluate whether these mAbs bind to linear and/or conformational epitopes through heat denaturation. hCA-IX fragments covering the entire hCA-IX ECD (FIG. 15B) are expressed as fusion proteins (Aga2-HA-(CA-IX)-MYC (pPNL6 vector) or (CA-IX)-Aga2-MYC (pPNL200 vector)) on the yeast cell surface. The epitope mapping of the mAbs was performed using a whole yeast cell ELISA protocol. Given that each hCA-IX fragment is expressed as a MYC fusion protein, the amount of properly displayed fusion protein can be assessed by probing with an anti-MYC antibody, followed by an HRP-conjugated secondary antibody. The anti-MYC signal can then used to normalize the binding signal for the 4A2 and 9B6 mAbs. These experiments were performed on native and denatured yeast cells, where in both cases binding of each mAb was normalized to the anti-MYC signal. The ratio of each normalized anti-CA-IX mAb signal of native versus denatured hCA-IX peptide is thus indicative of the conformational nature of the mAb binding epitope. In the native hCA-IX experiments the commercial M75 mAb was used as a positive control; the epitope of this mAb is known (Zavada et al., 2000) and is located in hCA-IX's PG domain. Table 10 (A, native rhCA-IX ECD; B, denatured rhCA-IX ECD) shows that the binding epitopes for the 4A2 and 9B6 mAbs are located in hCA-IX's catalytic domain whereas, as expected, that of the M75 mAb is located in hCA-IX's PG-like domain and confirms the PepScan observations. This data set also indicates that the 4A2 mAb epitope is likely to be structured, given the observation that binding is lost when the hCA-IX protein fragments are denatured.

TABLE 10
Results of the epitope mapping experiments by YSD of the recombinantly expressed mAbs 4A2 and
9B6 on either native or denatured peptides covering the hCA-IX ECD (−, no binding; +, binding)

| | | | | | | Anti-CA-IX Ab | | |
|---|---|---|---|---|---|---|---|---|
| | | | | | | binding intensity | | |
| | | | | | | (normalized on CA-IX_MYC | | |
| | | | | CA-IX | | expression on cells) | | |
| | YSD | | CA-IX | amino | CA-IX | | | M75 |
| Clone # | vector | Fused protein | Fragment # | acids | Domain | 4A2 | 9B6 | control |

A. Native CA-IX protein

| | | | | | | | | |
|---|---|---|---|---|---|---|---|---|
| 1A | PNL6 | Aga2-HA-CA9- | 1 | 52-111 | PG | − | − | ++ |
| | | MYC | | | | | | |
| 2B | PNL6 | Aga2-HA-CA9- | 2 | 38-136 | +PG+ | − | − | ++ |
| | | MYC | | | | | | |
| 3A | PNL6 | Aga2-HA-CA9- | 3 | 1-136 | SP+PG+ | − | − | ++++ |
| | | MYC | | | | | | |
| 4A | PNL6 | Aga2-HA-CA9- | 4 | 135-391 | CA | ++ | ++++ | − |
| | | MYC | | | | | | |
| 5A | PNL6 | Aga2-HA-CA9- | 5 | 112-391 | +CA | +++ | ++ | − |
| | | MYC | | | | | | |
| 6B | PNL6 | Aga2-HA-CA9- | 6 | 135-414 | CA+ | ++ | +++ | − |
| | | MYC | | | | | | |
| 7B | PNL6 | Aga2-HA-CA9- | 7 | 112-414 | +CA+ | ++++ | ++ | − |
| | | MYC | | | | | | |
| 8B | PNL6 | Aga2-HA-CA9- | 8 | 38-414 | +PG+CA+ | + | ++++ | +++ |
| | | MYC | | | | | | |
| 9A | PNL6 | Aga2-HA-CA9- | 9 | 1-414 | SP+PG+CA+ | Low | Low | ++++ |
| | | MYC | | | | level | level | |
| | | | | | | display | display | |
| | | | | | | of CA9 | of CA9 | |
| 11B | PNL6 | Aga2-HA-X- | Plasmid | Neg Ctrl | — | − | − | − |
| | | MYC | Ctrl | | | | | |
| EBY100 | No- | None | Strain Ctrl | Neg Ctrl | — | − | − | − |
| | plasmid | | | | | | | |
| 12B | PNL200 | CA9-Aga2- | 1 | 52-111 | PG | − | − | No data |
| | | MYC | | | | | | |
| 13A | PNL200 | CA9-Aga2- | 2 | 38-136 | +PG+ | − | − | No data |
| | | MYC | | | | | | |
| 14A | PNL200 | CA9-Aga2- | 3 | 1-136 | SP+PG+ | − | − | No data |
| | | MYC | | | | | | |
| 15B | PNL200 | CA9-Aga2- | 4 | 135-391 | CA | ++ | ++++ | No data |
| | | MYC | | | | | | |
| 16B | PNL200 | CA9-Aga2- | 5 | 112-391 | +CA | ++ | +++ | No data |
| | | MYC | | | | | | |
| 17B | PNL200 | CA9-Aga2- | 6 | 135-414 | CA+ | ++++ | ++ | No data |
| | | MYC | | | | | | |
| 18A | PNL200 | CA9-Aga2- | 7 | 112-414 | +CA+ | +++++ | ++ | No data |
| | | MYC | | | | | | |
| 19B | PNL200 | CA9-Aga2- | 8 | 38-414 | +PG+CA+ | ++++ | ++ | No data |
| | | MYC | | | | | | |
| 20A | PNL200 | CA9-Aga2- | 9 | 1-414 | SP+PG+CA+ | Low | ++++ | No data |
| | | MYC | | | | level | | |
| | | | | | | display | | |
| | | | | | | of CA9 | | |
| 22A | PNL200 | X-Aga2-MYC | Plasmid | Neg Ctrl | — | − | − | No data |
| | | | Ctrl | | | | | |
| EBY100 | No- | None | Strain Ctrl | Neg Ctrl | — | − | − | No data |
| | plasmid | | | | | | | |

B. Denatured CA-IX protein

| | | | | | | | | |
|---|---|---|---|---|---|---|---|---|
| 1A | PNL6 | Aga2-HA-CA9- | 1 | 52-111 | PG | − | − | − |
| | | MYC | | | | | | |
| 2B | PNL6 | Aga2-HA-CA9- | 2 | 38-136 | +PG+ | − | − | − |
| | | MYC | | | | | | |
| 3A | PNL6 | Aga2-HA-CA9- | 3 | 1-136 | SP+PG+ | − | − | − |
| | | MYC | | | | | | |
| 4A | PNL6 | Aga2-HA-CA9- | 4 | 135-391 | CA | − | ++ | − |
| | | MYC | | | | | | |
| 5A | PNL6 | Aga2-HA-CA9- | 5 | 112-391 | +CA | − | ++ | − |
| | | MYC | | | | | | |
| 6B | PNL6 | Aga2-HA-CA9- | 6 | 135-414 | CA+ | − | ++ | − |
| | | MYC | | | | | | |
| 7B | PNL6 | Aga2-HA-CA9- | 7 | 112-414 | +CA+ | − | +++ | − |
| | | MYC | | | | | | |
| 8B | PNL6 | Aga2-HA-CA9- | 8 | 38-414 | +PG+CA+ | − | +++ | − |
| | | MYC | | | | | | |
| 9A | PNL6 | Aga2-HA-CA9- | 9 | 1-414 | SP+PG+CA+ | − | Low | − |
| | | MYC | | | | | level | |
| | | | | | | | display | |
| | | | | | | | of CA9 | |
| 11B | PNL6 | Aga2-HA-X- | Plasmid | Neg Ctrl | — | − | − | − |
| | | MYC | Ctrl | | | | | |
| EBY100 | No- | None | Strain Ctrl | Neg Ctrl | — | − | − | − |
| | plasmid | | | | | | | |
| 12B | PNL200 | CA9-Aga2- | 1 | 52-111 | PG | − | − | − |
| | | MYC | | | | | | |
| 13A | PNL200 | CA9-Aga2- | 2 | 38-136 | +PG+ | − | − | − |
| | | MYC | | | | | | |
| 14A | PNL200 | CA9-Aga2- | 3 | 1-136 | SP+PG+ | − | − | − |
| | | MYC | | | | | | |
| 15B | PNL200 | CA9-Aga2- | 4 | 135-391 | CA | − | +++ | − |
| | | MYC | | | | | | |
| 16B | PNL200 | CA9-Aga2- | 5 | 112-391 | +CA | − | +++ | − |
| | | MYC | | | | | | |
| 17B | PNL200 | CA9-Aga2- | 6 | 135-414 | CA+ | − | +++ | − |
| | | MYC | | | | | | |
| 18A | PNL200 | CA9-Aga2- | 7 | 112-414 | +CA+ | − | ++++ | − |
| | | MYC | | | | | | |
| 19B | PNL200 | CA9-Aga2- | 8 | 38-414 | +PG+CA+ | − | ++++ | − |
| | | MYC | | | | | | |
| 20A | PNL200 | CA9-Aga2- | 9 | 1-414 | SP+PG+CA+ | − | Low | − |
| | | MYC | | | | | level | |
| | | | | | | | display | |
| | | | | | | | of CA9 | |
| 22A | PNL200 | X-Aga2-MYC | Plasmid | Neg Ctrl | — | − | − | − |
| | | | Ctrl | | | | | |
| EBY100 | No- | None | Strain Ctrl | Neg Ctrl | — | − | − | − |
| | plasmid | | | | | | | |

### Epitope Binding by SPR

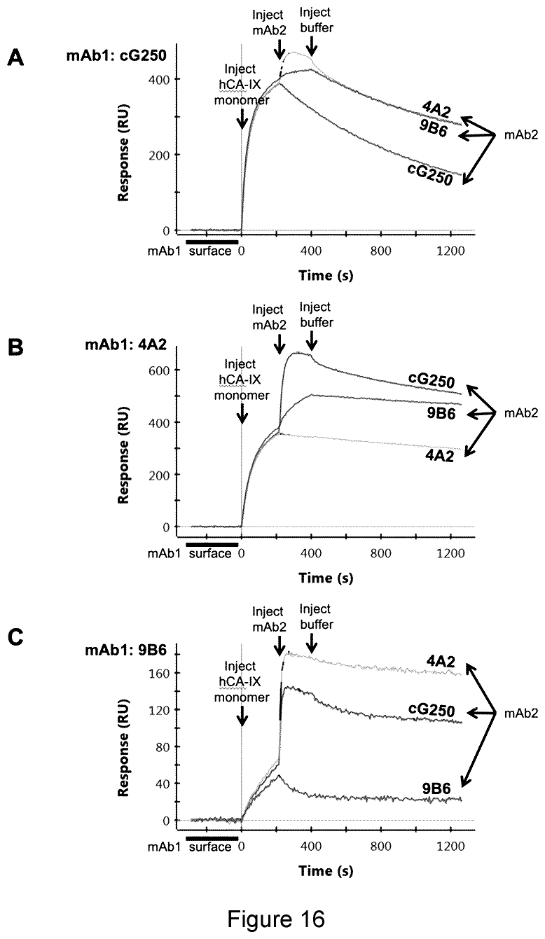

To confirm that the recombinantly produced 4A2 and 9B6 mAbs behave as the hybridoma produced mAbs in the epitope binding experiments, we repeated these experiments with the rhCA-IX ECD monomer (FIG. 16). A comparison of the off-rate of the hybridoma-derived (mouse scaffold) and recombinantly expressed (in a mouse IgG2b scaffold) 4A2 and 9B6 mAbs can be found in Table 11 and shows that the kinetics of the original hybridomas have been preserved in the chimeric mAbs. In addition, the cG250 mAb was included comparison of the data in the cG250 patent (WO2009/056342 A1) suggests that its binding epitope may overlap with that of the 9B6 mAb. The data presented here shows that neither the 9B6 nor the 4A2 mAb competes with cG250 for binding to hCA-IX indicating they interact with distinct binding epitopes. In addition, this data confirms that the 4A2 and the 9B6 mAb do not compete for binding to hCA-IX.

TABLE 11
Comparison of the SPR data generated with the hybridima-
derived and recombinantly expressed 4A2 and 9B6 mAbs

| Hybridoma-derived | | | | Recombinantly expressed | | | | |
|---|---|---|---|---|---|---|---|---|
| | | | rhCA-IX | | | | rhCA-IX | |
| Fusion | Clone | Scaffold | ECD | off rate | Mab | Scaffold | ECD | off rate |
| F117 | 4A2 | IgG2b | dimer | slow | 4A2 | mIgG2b | dimer | slow |
| F101 | 9B6 | IgG2b | monomer | slow | 9B6 | mIgG2b | monomer | slow |

### Example 6—Additional Functional Characterization

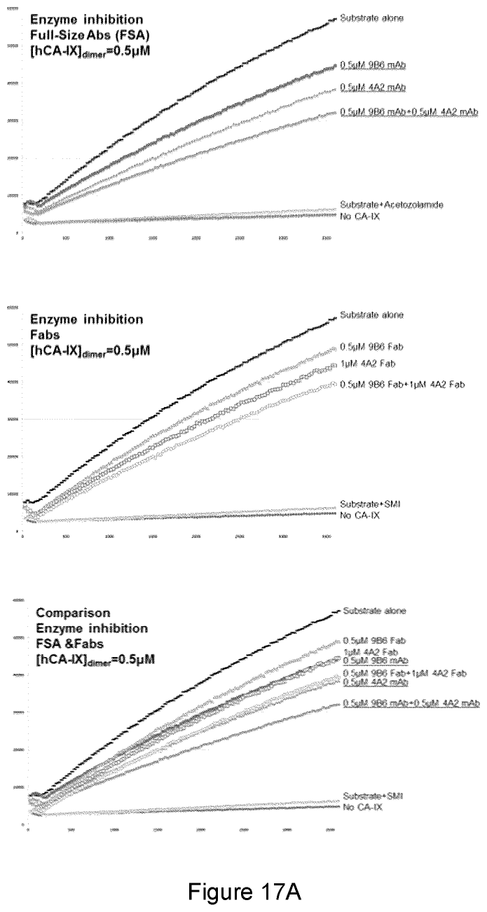

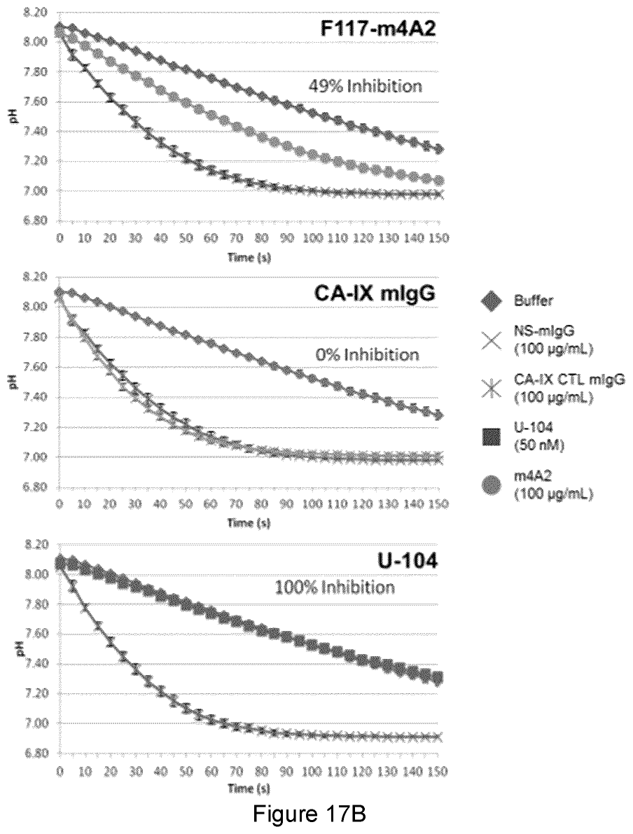

Since we determined that the hybridoma-derived 4A2 and 9B6 both inhibit the hCA-IX enzyme activity, to ˜60% and 40% respectively, and since both mAbs bind to distinguishable epitopes in the catalytic domain (shown by PepScan and SPR), we retested the recombinant mAbs but also their respective Fabs alone and as a combination in the enzyme activity assay. FIG. 17A shows that 0.5 μM mAb 4A2 and 9B6 alone inhibit the enzyme activity of the rhCA-IX dimer by 28% and 40% respectively whereas the 4A2+9B6 combo (0.5 μM+0.5 μM) results in a 50% inhibition. For the Fabs, a similar trend can be observed, i.e. the Fab combo inhibits the CA-IX enzyme (4A2+9B6 Fab=32%) to a greater extend than each of the Fabs alone (4A2Fab=22%, 9B6Fab=24). Comparison of the mAbs and Fab data shows however that the mAbs perform better than the Fabs.

These results prompted us to test the mAb 4A2 in a cell-based CA-IX activity assay. The major variation in this assay is the use of hCA-IX expressing 4T1 cells (67NR/hCA-IX cells; Lou et al., 2011) as the enzyme source instead of purified rhCA-IX. In addition, this assay uses water saturated with hCA-IX's natural substrate CO₂ instead of 4-NPA.

Briefly, subconfluent plates of 67NR/hCA-X cells were trypsinized for less than 5 minutes, cells were counted, spinned down and resuspended at 3.75×10⁶ cells in 1 ml of buffer (130 mM NaCl, 5 mM KCl, 20 mM Hepes, pH 8.0, 4° C.). 200 μl of buffer (7.5×10⁵ cells in total) were incubated for 50 min at room temperature (constant stirring) either alone or in the presence of antibody/IgG control (35.7 μg/ml). After the incubation period, buffer was added to a total volume of 800 μl, and 200 μl of CO₂-srated water were added to initiate the reaction. Substrate conversion was monitored every 5 sec as a drop in the pH. This method is based on the electrometric method of Wilbur and Anderson (1948; http://www.worthington-biochem.com/ca/assay.html), in which the time required (in seconds) for a saturated CO₂ solution to lower the pH of 0.012 M Tris-HCl buffer from 8.3 to 6.3 at 0° C. is determined. FIG. 17B shows that the 4A2 antibody inhibits the activity of cellularly expressed CA-IX by 49%, while the NS-mIgG and the CA-IX mIgG CTL had no effect.

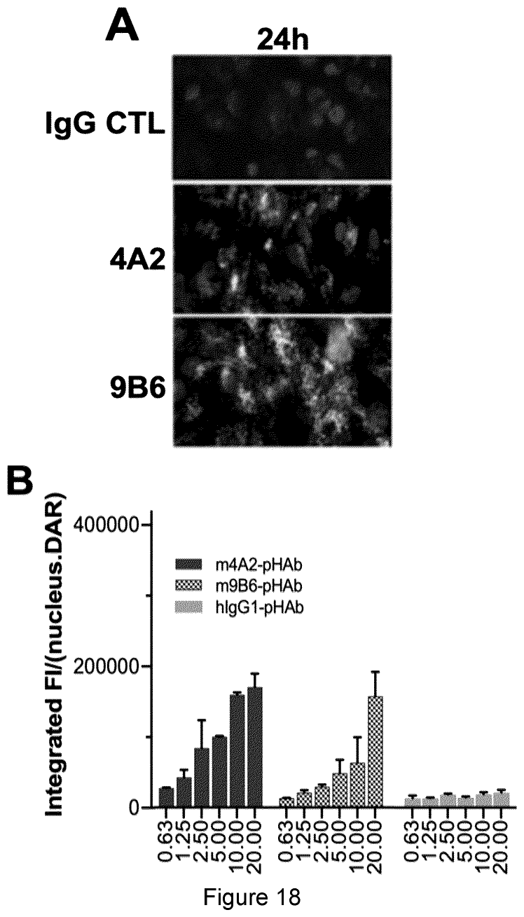

In addition, we also evaluated the internalization potential of these mAbs with the thought of using these enzyme-inhibiting mAbs to bring a toxic pay-load into cancer cells. For this, both mAbs, in addition to a negative control IgG, were labeled with a pH sensitive reactive dye (pHAb; Promega). Upon receptor-mediated internalization, these antibody-pHAb Dye conjugates traffick to the endosome and lysosomal vesicles where pH is acidic, causing the pHAb dye to fluoresce. CA-IX expressing SK-RC-52 expressing cells were incubated with these antibody-pHAb dye conjugates (10 μg/mL) and incubated at 37° C. (5% CO₂, humidified environment) for 24 h (FIG. 18A) or coated with various concentrations of the conjugates for 30 min at 4° C., washed, and then transferred to 37° C. (5% CO₂, humidified environment) for 24 h (FIG. 18B). Both experiments indicate that the 4A2 and 9B6 mAbs internalize in a CA-IX specific manner but that mAb 4A2 internalizes to a better extent that the 9B6 mAb. The latter may be due to the binding characteristics of 4A2 which is shown to have much better kinetic profile compared to 9B6 (FIG. 12).

The consistency of this data strongly suggests that these mAbs have the potential to become the next biologics for the treatment of renal and possibly other types of cancer.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

All publications and patent applications cited in this specification are herein incorporated by reference as if each individual publication or patent application were specifically and individually indicated to be incorporated by reference.

TABLE OF SEQUENCES

| | | SEQ ID |
|---|---|---|
| Definition | Sequence | NO. |
| CA-IX ECD | Exemplary Signal peptide-hCA-IX | 1 |
| | MAPLCPSPWLPLLIPAPAPGLTVQLLLSLLLLVPVHPQRLPRM | |
| | QEDSPLGGGSSGEDDPLGEEDLPSEEDSPREEDPPGEEDLP | |
| | GEEDLPGEEDLPEVKPKSEEEGSLKLEDLPTVEAPGDPQEPQ | |
| | NNAHRDKEGDDQSHWRYGGDPPWPRVSPACAGRFQSPVDI | |
| | RPQLAAFCPALRPLELLGFQLPPLPELRLRNNGHSVQLTLPP | |
| | GLEMALGPGREYRALQLHLHWGAAGRPGSEHTVEGHRFPA | |
| | EIHVVHLSTAFARV [symbol] ALGRPGGLAVLAAFLEEGPEENSAYE | |
| | QLLSRLEEIAEEGSETQVPGLDISALLPSDFSRYFQYEGSLTT | |
| | PPCAQGVIWTVFNQTVMLSAKQLHTLSDTLWGPGDSRLQLN | |
| | FRATQPLNGRVIEASFPAGVDSSPRAAEPVQLNSCLAAGDGS | |
| | HHHHHHHHHHG | |
| Putative CA-IX | DQSHW | 2 |
| peptide epitope of | | |
| 4A2 | | |
| CA-IX peptide | DEALGR | 3 |
| epitope of 4A2 | | |
| CA-IX peptide | STAFARVDE | 4 |
| epitope of 9B6 | | |
| Overlapping | STAFARVDEALGR | 5 |
| peptide of | | |
| CA-IX epitope | | |

F117-m4A2 antibody V_{H} and V_{L} CDR sequences

| | | |
|---|---|---|
| Light chain: | Leader sequence-FR1-CDR1-FR2-CDR2-FR3-CDR3-FR4 | 6 |
| Amino acids | MDFQVQIFSFLLISASVILSRGQIVLTQSPAVMSAFPGEKVTM | |
| sequence (128 | TCSASSSVGYMHWYQQKAGSSPRLLIYDTSNLSSGVPARCS | |
| AA) | GSGSGTSYSLTISRMEAEDAATYYCQQWRSYPPTFGGGTKL | |
| | EIK | |
| Light chain: | SASSSVGYMH | 7 |
| CDR1 (L1) | | |
| Light chain: | DTSNLSS | 8 |
| CDR2 (L2) | | |
| Light chain: | QQWRSYPPT | 9 |
| CDR3 (L3) | | |
| Heavy chain: | Leader sequence-FR1-CDR1-FR2-CDR2-FR3-CDR3-FR4 | 10 |
| Amino acids | MYLGLNCVFIVFLLKGVQSEVKLEESGGGLVQPGRSMKLSC | |
| sequence (140 | VASGFTFSYYWMDWVRQSPEKGLEWVAEIRLKSDNYATHY | |
| AA) | AESVKGRFTISRDDSKSSVYLQMNNLRAEDTGIYYCTRAPHY | |
| | YGYFDYWGQGTTLTVSS | |
| Heavy chain: | GFTFSYYWMD | 11 |
| CDR1 (H1) | | |
| Heavy chain: | EIRLKSDNYATHYAESVKG | 12 |
| CDR2 (H2) | | |
| Heavy chain: | APHYYGYFDY | 13 |
| CDR3 (H3) | | |

F101-m9B6 antibody V_{H} and V_{L} CDR sequence

| | | |
|---|---|---|
| Light chain: | Leader sequence-FR1-CDR1-FR2-CDR2-FR3-CDR3-FR4 | 14 |
| Amino acids | METDTILLWVLLLWVPGSTGDIVLTQSPSSLAVSLGQRATISC | |
| sequence (131 | KASQSVDYDGNSYMNWFQQKPGQPPKLLIYEASSLESGIPA | |
| AA) | RISGSGSGTDFTLNIHPVEEEDAATYYCQQSYEGPYTFGGG | |
| | TKLEIK | |
| Light chain | KASQSVDYDGNSYMN | 15 |
| CDR1 (L1) | | |
| Light chain | EASSLES | 16 |
| CDR2 (L2) | | |
| Light chain | QQSYEGPYT | 17 |
| CDR3 (L3) | | |
| Heavy chain: | Leader sequence-FR1-CDR1-FR2-CDR2-FR3-CDR3-FR4 | 18 |
| Amino acids | MGWSCLILFLVAAATGVHSQVQLQQPGAELVKPGASVKLSC | |
| sequence (132 | KASGYIFTTKWINWVKQRPGQGLEWIGNIYPGSSNTYYNEKF | |
| AA) | KNKATLTVDKSSNTAHLQLSSLTSEDSAVYYCARGIANWGQ | |
| | GTPVTVSA | |
| Heavy chain: | GYIFTTKWIN | 19 |
| CDR1 (H1) | | |
| Heavy chain: | NIYPGSSNTYYNEKFKN | 20 |
| CDR2 (H2) | | |
| Heavy chain: | GIAN | 21 |
| CDR3 (H3) | | |

RECOMBINANT ANTIBODIES

| | | |
|---|---|---|
| 4A2 light chain | Exemplary Signal peptide - Light chain | 22 |
| (mouse κ1) | MVLQTQVFISLLLWISGAYGQIVLTQSPAVMSAFPGEKVTMTC | |
| | SASSSVGYMHWYQQKAGSSPRLLIYDTSNLSSGVPARCSGS | |
| | GSGTSYSLTISRMEAEDAATYYCQQWRSYPPTFGGGTKLEIK | |
| | RADAAPTVSIFPPSSEQLTSGGASVVCFLNNFYPKDINVKWKI | |
| | DGSERQNGVLNSWTDQDSKDSTYSMSSTLTLTKDEYERHNS | |
| | YTCEATHKTSTSPIVKSFNRNEC | |
| 4A2 heavy chain | Exemplary Signal peptide - Heavy chain | 23 |
| (mouse | MDWTWRILFLVAAATGTHAEVKLEESGGGLVQPGRSMKLSC | |
| IgG2b) | VASGFTFSYYWMDWVRQSPEKGLEWVAEIRLKSDNYATHYA | |
| | ESVKGRFTISRDDSKSSVYLQMNNLRAEDTGIYYCTRAPHYY | |
| | GYFDYWGQGTTLTVSSAKTTPPSVYPLAPGCGDTTGSSVTL | |
| | GCLVKGYFPESVTVTWNSGSLSSSVHTFPALLQSGLYTMSSS | |
| | VTVPSSTWPSQTVTCSVAHPASSTTVDKKLEPSGPISTINPCP | |
| | PCKECHKCPAPNLEGGPSVFIFPPNIKDVLMISLTPKVTCVVV | |
| | DVSEDDPDVQISWFVNNVEVHTAQTQTHREDYNSTIRVVSTL | |
| | PIQHQDWMSGKEFKCKVNNKDLPSPIERTISKIKGLVRAPQVY | |
| | ILPPPAEQLSRKDVSLTCLVVGFNPGDISVEWTSNGHTEENY | |
| | KDTAPVLDSDGSYFIYSKLNMKTSKWEKTDSFSCNVRHEGLK | |
| | NYYLKKTISRSPG | |
| 9B6 Light chain | Exemplary Signal peptide - Light chain | 24 |
| (mouse κ1) | MVLQTQVFISLLLWISGAYGDIVLTQSPSSLAVSLGQRATISCK | |
| | ASQSVDYDGNSYMNWFQQKPGQPPKLLIYEASSLESGIPARI | |
| | SGSGSGTDFTLNIHPVEEEDAATYYCQQSYEGPYTFGGGTKL | |
| | EIKRADAAPTVSIFPPSSEQLTSGGASVVCFLNNFYPKDINVK | |
| | WKIDGSERQNGVLNSWTDQDSKDSTYSMSSTLTLTKDEYER | |
| | HNSYTCEATHKTSTSPIVKSFNRNEC | |
| 9B6 Heavy chain | Exemplary Signal peptide - Light chain | 25 |
| (mouse IgG2b) | MDWTWRILFLVAAATGTHAQVQLQQPGAELVKPGASVKLSC | |
| | KASGYIFTTKWINWVKQRPGQGLEWIGNIYPGSSNTYYNEKF | |
| | KNKATLTVDKSSNTAHLQLSSLTSEDSAVYYCARGIANWGQG | |
| | TPVTVSAAKTTPPSVYPLAPGCGDTTGSSVTLGCLVKGYFPE | |
| | SVTVTWNSGSLSSSVHTFPALLQSGLYTMSSSVTVPSSTWPS | |
| | QTVTCSVAHPASSTTVDKKLEPSGPISTINPCPPCKECHKCPA | |
| | PNLEGGPSVFIFPPNIKDVLMISLTPKVTCVVVDVSEDDPDVQI | |
| | SWFVNNVEVHTAQTQTHREDYNSTIRVVSTLPIQHQDWMSG | |
| | KEFKCKVNNKDLPSPIERTISKIKGLVRAPQVYILPPPAEQLSR | |
| | KDVSLTCLVVGFNPGDISVEWTSNGHTEENYKDTAPVLDSDG | |
| | SYFIYSKLNMKTSKWEKTDSFSCNVRHEGLKNYYLKKTISRSP | |
| | G | |
| CDR L1-3 of 4A2 | SASSSVGYMHDTSNLSSQQWRSYPPT | 26 |
| CDR L1-3 of 9B6 | KASQSVDYDGNSYMNEASSLESQQSYEGPYT | 27 |
| CDR H1-3 of 4A2 | GFTFSYYWMDEIRLKSDNYATHYAESVKGAPHYYGYFDY | 28 |
| CDR H1-3 of 9B6 | GYIFTTKWINEIRLKSDNYATHYAESVKGAPHYYGYFDY | 29 |
| Consensus | X₁ASX₂SVX₃X₄X₅X₆X₇X₈YMX₉X₁₀X₁₁SX₁₂LX₁₃SQQX₁₄X₁₅X₁₆X₁₇ | 30 |
| sequence | PX₁₈T | |
| Light chain | wherein X₁ is S or K, X₂ is S or Q, X₃ is D or no amino acid, X₄ is | |
| CDR 1-3 (L1-3) | Y or no amino acid, X₅ is D or no amino acid, X₆ is G or no | |
| | amino acid, X₇ is N or no amino acid, X₈ is G or S, X₉ is H or N, | |
| | X₁₀ is D or E, X₁₁ is T or A, X₁₂ is N or S, X₁₃ is S or E, X₁₄ is W | |
| | or S, X₁₅ is R or Y, X₁₆ S or E, X₁₇ Y or G and PX₁₈ is P or Y | |
| Consensus | GX₂₁X₂₂FX₂₃X₂₄X₂₅WX₂₆X₂₇EIRLKSDNYATHYAESVKGAPHYY | 31 |
| sequence | GYFDY | |
| Heavy chain | wherein X₂₁ is F or Y, X₂₂ is T or I, X₂₃ is S or T, X₂₄ is Y or T, | |
| CDR 1-3 (H1-3) | X₂₅ is Y or K, X₂₆ is M or I, X₂₇ is D or N | |
| Light chain | X₁ASX₂SVX₃X₄X₅X₆X₇X₈YMX₉ | 32 |
| Consensus | wherein X₁ is S or K, X₂ is S or Q, X₃ is D or no amino acid, X₄ is | |
| CDR1 (L1) | Y or no amino acid, X₅ is D or no amino acid, X₆ is G or no | |
| | amino acid, X₇ is N or no amino acid, X₈ is G or S, X₉ is H or N | |
| Light chain | X₁₀X₁₁SX₁₂LX₁₃S | 33 |
| Consensus | wherein X₁₀ is D or E, X₁₁ is T or A, X₁₂ is N or S, X₁₃ is S or E | |
| CDR2 (L2 | | |
| Light chain | QQX₁₄X₁₅X₁₆X₁₇ PX₁₈T | 34 |
| Consensus | wherein X₁₄ is W or S, X₁₅ is R or Y, X₁₆ S or E, X₁₇ Y or G and | |
| CDR3 (L3) | PX₁₈ is P or Y | |
| Heavy chain | GX₂₁X₂₂FX₂₃X₂₄X₂₅WX₂₆X₂₇ | 35 |
| Consensus | wherein X₂₁ is F or Y, X₂₂ is T or I, X₂₃ is S or T, X₂₄ is Y or T, | |
| CDR1 (H1) | X₂₅ is Y or K, X₂₆ is M or I, X₂₇ is D or N | |
| Heavy chain | EIRLKSDNYATHYAESVKGA | 36 |
| Consensus | | |
| CDR2 (H2) | | |
| Heavy chain | PHYYGYFDY | 37 |
| Consensus | | |
| CDR3 (H3) | | |

DNA sequences

| | | |
|---|---|---|
| 4A2 | Light chain: DNA sequence (384 bp) | 38 |
| | Leader sequence-FR1-CDR1-FR2-CDR2-FR3-CDR3-FR4 | |
| | ATGGATTTTCAAGTGCAGATTTTCAGCTTCCTGCTAATCAG | |
| | TGCCTCAGTCATACTGTCCAGAGGACAAATTGTTCTCACC | |
| | CAGTCTCCAGCAGTCATGTCTGCATTTCCAGGGGAGAAG | |
| | GTCACCATGACCTGCAGTGCCAGCTCAAGTGTAGGTTACA | |
| | TGCACTGGTACCAGCAGAAGGCAGGATCGTCCCCCAGAC | |
| | TCCTGATTTATGACACATCCAATCTGTCTTCTGGAGTCCCT | |
| | GCTCGCTGCAGTGGCAGTGGGTCTGGGACCTCTTACTCT | |
| | CTCACAATCAGCCGAATGGAGGCTGAAGATGCTGCCACTT | |
| | ATTACTGCCAGCAGTGGAGGAGTTACCCACCCACGTTCG | |
| | GAGGGGGGACCAAGCTGGAAATAAAA | |
| 4A2 | Heavy chain: DNA sequence (420 bp) | 39 |
| | Leader sequence-FR1-CDR1-FR2-CDR2-FR3-CDR3-FR4 | |
| | ATGTACTTGGGACTGAACTGTGTATTCATAGTTTTTCTCTT | |
| | AAAAGGTGTCCAGAGTGAAGTGAAGCTTGAGGAGTCTGG | |
| | AGGAGGCTTGGTGCAACCTGGAAGATCCATGAAACTCTCC | |
| | TGTGTTGCCTCTGGATTCACTTTCAGTTACTACTGGATGGA | |
| | CTGGGTCCGCCAGTCTCCAGAGAAGGGGCTTGAGTGGGT | |
| | TGCTGAAATTAGATTGAAGTCTGATAATTATGCAACACATT | |
| | ATGCGGAGTCTGTGAAAGGGAGGTTCACCATCTCAAGAG | |
| | ATGATTCCAAAAGTAGTGTCTACCTGCAAATGAACAACTTA | |
| | AGAGCTGAAGACACTGGCATTTATTACTGTACCAGGGCGC | |
| | CTCATTACTATGGCTACTTTGACTACTGGGGCCAAGGCAC | |
| | CACTCTCACAGTCTCCTCA | |
| 9B6 | Light chain: DNA sequence (393 bp) | 40 |
| | Leader sequence-FR1-CDR1-FR2-CDR2-FR3-CDR3-FR4 | |
| | ATGGAGACAGACACAATCCTGCTATGGGTGCTGCTGCTCT | |
| | GGGTTCCAGGCTCCACTGGTGACATTGTGCTGACCCAATC | |
| | TCCATCTTCTTTGGCTGTGTCTCTAGGGCAGAGGGCCACC | |
| | ATCTCCTGCAAGGCCAGCCAAAGTGTTGATTATGATGGTA | |
| | ATAGTTATATGAACTGGTTCCAACAGAAACCAGGACAGCC | |
| | ACCCAAACTCCTCATCTATGAAGCATCCAGTCTAGAATCT | |
| | GGAATCCCAGCCAGGATTAGTGGCAGTGGGTCTGGGACA | |
| | GACTTCACCCTCAACATCCATCCTGTGGAGGAGGAGGAT | |
| | GCTGCAACCTATTACTGTCAGCAAAGTTATGAGGGTCCGT | |
| | ACACGTTCGGAGGGGGGACCAAGCTGGAAATAAAA | |
| 9B6 | Heavy chain: DNA sequence (396 bp) | 41 |
| | Leader sequence-FR1-CDR1-FR2-CDR2-FR3-CDR3-FR4 | |
| | ATGGGATGGAGCTGTCTCATCCTCTTTTTGGTAGCTGCAG | |
| | CTACAGGTGTCCACTCCCAGGTCCAACTGCAGCAGCCTG | |
| | GGGCTGAGCTTGTGAAGCCTGGGGCTTCAGTGAAGTTGT | |
| | CCTGCAAGGCTTCTGGCTACATTTTCACCACCAAGTGGAT | |
| | AAACTGGGTGAAGCAGAGGCCTGGACAAGGCCTTGAATG | |
| | GATTGGAAATATTTATCCTGGTAGTAGCAATACTTACTACA | |
| | ATGAGAAATTCAAGAACAAGGCTACACTGACTGTGGACAA | |
| | ATCCTCCAACACAGCCCACTTGCAGCTCAGCAGCCTGACA | |
| | TCTGAGGACTCTGCGGTCTATTATTGTGCAAGAGGGATTG | |
| | CTAACTGGGGCCAAGGGACTCCGGTCACTGTCTCTGCA | |
| 4A2 VL | SASSSV-----GYMHDTSNLSSQQWRSYPPT | 42 |
| CDR1-3 | | |
| 9B6 VL | KASQSVDYDGNSYMNEASSLESQQSYEGPYT | 43 |
| CDR 1-3 | | |
| 4A2 VL | GFTFSYYWMDEIRLKSDNYATHYAESVKGAPHYYGYFDY | 44 |
| CDR1-3 | | |
| 9B6 VH | GYIFTTKWINEIRLKSDNYATHYAESVKGAPHYYGYFDY | 45 |
| CDR1-3 | | |
| Minimum epitope | RVDEAL | 46 |
| sequence | | |

### REFERENCES

Ahlskog J K, Schliemann C, Marlind J, Qureshi U, Ammar A, Pedley R B, Neri D. Human monoclonal antibodies targeting carbonic anhydrase IX for the molecular imaging of hypoxic regions in solid tumours. Br J Cancer. 2009 Aug. 18; 101(4):645-57.
Alterio V, Hilvo M, Di Fiore A, Supuran C T, Pan P, Parkkila S, Scaloni A, Pastorek J, Pastorekova S, Pedone C, Scozzafava A, Monti S M, De Simone G. Crystal structure of the catalytic domain of the tumor-associated human carbonic anhydrase IX. Proc Natl Acad Sci USA. 2009 Sep. 22; 106(38): 16233-8.
Antal I, Koneracka M, Kubovcikova M, Zavisova V, Khmara I, Lucanska D, Jelenska L, Vidlickova I. Zatovicova M, Pastorekova S, Bugarova N, Micusik M, Omastova M, Kopcansky P. d,l-lysine functionalized Fe304 nanoparticles for detection of cancer cells. Colloids Surf B Biointerfaces. 2018 Mar. 1; 163:236-245.
Brouwers A, Mulders P, Oosterwijk E, Buijs W, Corstens F, Boerman O, Oyen W. Pharmacokinetics and tumor targeting of 131 l-labeled F(ab′)2 fragments of the chimeric monoclonal antibody G250: preclinical and clinical pilot studies. Cancer Biother Radiopharm. 2004 August; 19(4):466-77.
Chrastina A, Závada J, Parkkila S, Kaluz S, Kaluzová M, Rajcani J, Pastorek J, Pastorekova S. Biodistribution and pharmacokinetics of 1251-labeled monoclonal antibody M75 specific for carbonic anhydrase IX, an intrinsic marker of hypoxia, in nude mice xenografted with human colorectal carcinoma. Int J Cancer. 2003a Jul. 20; 105(6):873-81.
Chrastina A, Pastoreková S, Pastorek J. Immunotargeting of human cervical carcinoma xenograft expressing CA IX tumor-associated antigen by 1251-labeled M75 monoclonal antibody. Neoplasma. 2003b; 50(1): 13-21.
Dereeper A, Guignon V, Blanc G, Audio S, Buffet S, Chevenet F, Dufayard J F, Guindon S, Lefort V, Lescot M, Claverie J M, Gascuel O. Phylogeny.fr: robust phylogenetic analysis for the non-specialist. Nucleic Acids Res. 2008 Jul. 1; 36 (Web Server issue):W465-9.
Dereeper A, Audio S, Claverie J M, Blanc G. BLAST-EXPLORER helps you building datasets for phylogenetic analysis. BMC Evol Biol. 2010 Jan. 12; 10:8.
Ditte P, Dequiedt F, Svastova E, Hulikova A, Ohradanova-Repic A, Zatovicova M, Csaderova L, Kopacek J, Supuran C T, Pastorekova S, Pastorek J. Phosphorylation of carbonic anhydrase X controls its ability to mediate extracellular acidification in hypoxic tumors. Cancer Res. 2011 Dec. 15; 71(24):7558-67.
Ebert T, Bander N H, Finstad C L, Ramsawak R D, Old L J. Establishment and characterization of human renal cancer and normal kidney cell lines. Cancer Res. 1990 Sep. 1; 50(17):5531-6.
Edgar R C. MUSCLE: a multiple sequence alignment method with reduced time and space complexity. BMC Bioinformatics. 2004 Aug. 19; 5:113.
Feldhaus M J, Siegel R W, Opresko L K, Coleman J R, Feldhaus J M, Yeung Y A, Cochran J R, Heinzelman P, Colby D, Swers J, Graff C, Wiley H S, Wittrup K D. Flow-cytometric isolation of human antibodies from a nonimmune Saccharomyces cerevisiae surface display library. Nat Biotechnol. 2003 February; 21 (2): 163-70.
Hulikova A, Zatovicova M, Svastova E, Ditte P, Brasseur R, Kettmann R, Supuran C T, Kopacek J, Pastorek J, Pastorekova S. Intact intracellular tail is critical for proper functioning of the tumor-associated, hypoxia-regulated carbonic anhydrase IX. FEBS Lett. 2009 Nov. 19; 583(22): 3563-8.
Lau J, Lin K, Benard, F. Past, Present, and Future: Development of Theranostic Agents Targeting Carbonic Anhydrase IX. Theranostics. 2017; 7(17): 4322-4339.
Lou Y, McDonald P C, Oloumi A, Chia S, Ostlund C, Ahmadi A, Kyle A, Auf dem Keller U, Leung S, Huntsman D, Clarke B, Sutherland B W, Waterhouse D, Bally M, Roskelley C, Overall C M, Minchinton A, Pacchiano F, Carta F, Scozzafava A, Touisni N, Winum J Y, Supuran C T, Dedhar S. Targeting tumor hypoxia: suppression of breast tumor growth and metastasis by novel carbonic anhydrase IX inhibitors. Cancer Res. 2011 May 1; 71(9):3364-76.
Mboge M Y, Mahon B P, McKenna R, Frost S C. Carbonic Anhydrases: Role in pH Control and Cancer. Metabolites. 2018 Feb. 28; 8(1).
Murri-Plesko M T, Hulikova A, Oosterwijk E, Scott A M, Zortea A, Harris A L, Ritter G, Old L, Bauer S, Swietach P, Renner C. Antibody inhibiting enzymatic activity of tumour-associated carbonic anhydrase isoform IX. Eur J Pharmacol. 2011 Apr. 25; 657(1-3): 173-83.
Neri D, Supuran C T. Interfering with pH regulation in tumours as a therapeutic strategy. Nat Rev Drug Discov. 2011 Sep. 16; 10(10):767-77.
Oosterwijk E, Boerman O C, Oyen W J, Old L J, Mulders P F. Antibody therapy in renal cell carcinoma. Monoclonal antibody G 250 recognizes a determinant present in renal-cell carcinoma and absent from normal kidney. World J Urol. 2008 April; 26(2): 141-6.
Pacchiano F, Aggarwal M, Avvaru B S, Robbins A H, Scozzafava A, McKenna R, Supuran C T. Selective hydrophobic pocket binding observed within the carbonic anhydrase II active site accommodate different 4-substituted-ureido-benzenesulfonamides and correlate to inhibitor potency. Chem Commun (Camb). 2010 Nov. 28; 46(44):8371-3.
Pastorekova S, Parkkila S, Pastorek J, Supuran C T. Carbonic anhydrases: current state of the art, therapeutic applications and future prospects. J Enzyme Inhib Med Chem. 2004 June; 19(3):199-229.
Petrul H M1, Schatz C A, Kopitz C C, Adnane L, McCabe T J, Trail P, Ha S, Chang Y S, Voznesensky A, Ranges G, Tamburini P P. Therapeutic mechanism and efficacy of the antibody-drug conjugate BAY79-4620 targeting human carbonic anhydrase 9. Mol Cancer Ther. 2012 February; 11(2):340-9.
Siebels M, Rohrmann K, Oberneder R, Stahler M, Haseke N, Beck J, Hofmann R, Kindler M, Kloepfer P, Stief C. A clinical phase I/II trial with the monoclonal antibody cG250 (RENCAREX®) and interferon-alpha-2a in metastatic renal cell carcinoma patients. World J Urol. 2011 February; 29(1):121-6.
Stillebroer A B, Boerman O C, Desar I M, Boers-Sonderen M J, van Herpen C M, Langenhuijsen J F, Smith-Jones P M, Oosterwijk E, Oyen W J, Mulders P F. Phase 1 radioimmunotherapy study with lutetium 177-labeled anti-carbonic anhydrase IX monoclonal antibody girentuximab in patients with advanced renal cell carcinoma. Eur Urol. 2013 September; 64(3):478-85.
Supuran C T, Di Fiore A, De Simone G. Carbonic anhydrase inhibitors as emerging drugs for the treatment of obesity. Expert Opin Emerg Drugs. 2008 June; 13(2):383-92.
Surfus J E, Hank J A, Oosterwijk E, Welt S, Lindstrom M J, Albertini M R, Schiller J H, Sondel P M. Anti-renal-cell carcinoma chimeric antibody G250 facilitates antibody-dependent cellular cytotoxicity with in vitro and in vivo interleukin-2-activated effectors. J Immunother Emphasis Tumor Immunol. 1996 May; 19(3):184-91.
Svastová E, Zilka N, Zat'ovicová M, Gibadulinová A, Ciampor F, Pastorek J, Pastoreková S. Carbonic anhydrase IX reduces E-cadherin-mediated adhesion of MDCK cells via interaction with beta-catenin. Exp Cell Res. 2003 Nov. 1; 290(2):332-45.
Svastová E, Witarski W, Csaderova L, Kosik I, Skvarkova L, Hulikova A, Zatovicova M, Barathova M, Kopacek J, Pastorek J, Pastorekova S. Carbonic anhydrase IX interacts with bicarbonate transporters in lamellipodia and increases cell migration via its catalytic domain. J Biol Chem. 2012 Jan. 27; 287(5):3392-402.
Thiry A, Dogne J M, Masereel B, Supuran C T. Targeting tumor-associated carbonic anhydrase IX in cancer therapy. Trends Pharmacol Sci. 2006 November; 27(11):566-73.
Timmerman_P, Barderas R, Desmet J, Altschuh D, Shochat S, Hollestelle M J, Höppener J W, Monasterio A, Casal J I, Meloen R H. A combinatorial approach for the design of complementarity-determining region-derived peptidomimetics with in vitro anti-tumoral activity. J Biol Chem. 2009 Dec. 4; 284(49):34126-34.
Ward C, Meehan J, Gray M, Kunkler I H, Langdon S P, Argyle D J. Carbonic Anhydrase IX (CAIX), Cancer, and Radiation Responsiveness. Metabolites. 2018 Feb. 10; 8(1).
WO2009/056342
Wykoff C C, Beasley N J, Watson P H, Turner K J, Pastorek J, Sibtain A, Wilson G D, Turley H, Talks K L, Maxwell P H, Pugh C W, Ratcliffe P J, Harris A L. Hypoxia-inducible expression of tumor-associated carbonic anhydrases. Cancer Res. 2000 Dec. 15; 60(24):7075-83.
Xu C, Lo A, Yammanuru A, Tallarico A S, Brady K, Murakami A, Barteneva N, Zhu Q, Marasco W A. Unique biological properties of catalytic domain directed human anti-CAIX antibodies discovered through phage-display technology. PLoS One. 2010 Mar. 10; 5(3):e9625.
Zatovicova M, Sedlakova O, Svastova E, Ohradanova A, Ciampor F, Arribas J, Pastorek J, Pastorekova S. Ectodomain shedding of the hypoxia-induced carbonic anhydrase IX is a metalloprotease-dependent process regulated by TACE/ADAM17. Br J Cancer. 2005 Nov. 28; 93(11): 1267-76.
Zatovicova M, Jelenska L, Hulikova A, Csaderova L, Ditte Z, Ditte P, Goliasova T, Pastorek J, Pastorekova S. (2010) Carbonic anhydrase IX as an anticancer therapy target: preclinical evaluation of internalizing monoclonal antibody directed to catalytic domain. Curr Pharm Des. 16(29):3255-63.
Závada J, Závadová Z, Pastoreková S, Ciampor F, Pastorek J, Zelnik V. Expression of MaTu-MN protein in human tumor cultures and in clinical specimens. Int J Cancer. 1993 May 8; 54(2):268-274.
Závada J, Závadová Z, Pastorek J, Biesová Z, Jezek J, Velek J. Human tumour-associated cell adhesion protein MN/CA IX: identification of M75 epitope and of the region mediating cell adhesion. Br J Cancer. 2000 June; 82(11):1808-13.

1. An antibody or a fragment thereof, wherein the antibody or fragment binds to Carbonic Anhydrase-IX (CA-IX) catalytic domain.

2. The antibody or fragment of claim 1, wherein the antibody or fragment thereof specifically binds to a peptide selected from the group consisting of: DEALGR (SEQ ID NO.3), STAFARVDE (SEQ ID NO.4), and STAFARVDEALGR (SEQ ID NO.5).

3. The antibody or fragment of claim 1 or 2, wherein the antibody or fragment thereof inhibits CA-IX catalytic activity in an enzymatic assay and in a cellular assay.

4. The antibody or fragment of any one of claims 1 to 4, wherein the antibody or fragment thereof inhibits CA-IX by at least 20% when used at a concentration of 0.5 μM in a cellular assay.

5. The antibody or fragment of any one of claims 1 to 3, wherein the antibody or fragment thereof inhibits CA-IX by at least 25%, when used at a concentration of 0.5 μM in an enzymatic assay.

6. The antibody or fragment of any one of claims 1 to 5, wherein the antibody or fragment thereof is isolated or purified.

7. An antibody or fragment thereof which comprises a light chain (L) comprising:
a complementarity determining region (CDR) 1 (L1) comprising the sequence X₁ASX₂SVX₃X₄X₅X₆X₇X₈YMX₉ wherein X₁ is S or K, X₂ is S or Q, X₃ is D or no amino acid, X₄ is Y or no amino acid, X₅ is D or no amino acid, X₆ is G or no amino acid, X₇ is N or no amino acid, X₈ is G or S, X₉ is H or N (SEQ ID NO.32);
a CDR2 (L2) comprising the sequence X₁₀X₁₁SX₁₂LX₁₃S wherein X₁₀ is D or E, Xu is T or A, X₁₂ is N or S, X₁₃ is S or E (SEQ ID NO.33); and
a CDR3 (L3) comprising the sequence QQX₁₄X₁₅X₁₆X₁₇PX₁₈T wherein X₁₄ is W or S, X₁₅ is R or Y, X₁₆ S or E, X₁₇ Y or G and PX₁₈ is P or Y (SEQ ID NO.34);
or a sequence substantially identical thereto;
wherein the antibody or fragment is specific for CA-IX.

8. An antibody or fragment thereof which comprises a heavy chain (H) comprising:
a complementarity determining region (CDR) 1 (H1) comprising a peptide defined by sequence: GX₂₁X₂₂FX₂₃X₂₄X₂₅WX₂₆X₂₇ wherein X₂₁ is F or Y, X₂₂ is T or I, X₂₃ is S or T, X₂₄ is Y or T, X₂₅ is Y or K, X₂₆ is M or I, X₂₇ is D or N (SEQ ID NO.35);
a CDR2 (H2) comprising a peptide defined by sequence: EIRLKSDNYATHY AESVKGA (SEQ ID NO.36); and
a CDR3 H3) comprising a peptide defined by sequence: PHYYGYFDY (SEQ ID NO.37);
or a sequence substantially identical thereto;
wherein the antibody or fragment is specific for CA-IX.

9. The antibody or fragment according to any one of claims 1 to 5, comprising:
a light chain comprising the CDRs L1-3 (SEQ ID NO.30) as defined in claim 7; and
a heavy chain comprising the CDRs H1-3 (SEQ ID NO.31) as defined claim 8;
or a sequence substantially identical thereto.

10. The antibody or fragment according to claim 7, wherein said CDR L1 is defined as:
(SEQ ID NO. 7)
SASSSVGYMH
or
(SEQ ID No. 15)
KASQSVDYDGNSYMN.

11. The antibody or fragment according to claim 7 or 10, wherein said CDR L2 is defined as:
(SEQ ID NO. 8)
DTSNLSS
or
(SEQ ID NO. 16)
EASSLES.

12. The antibody or fragment according to any one of claims 7, 10 and 11, wherein said CDR L3 is defined as: QQWRSYPPT (SEQ ID NO.9) or QQSYEGPYT (SEQ ID NO. 17)

13. The antibody or fragment according to claim 8, wherein said CDR H1 is defined as:
(SEQ ID NO. 11)
GFTFSYYWMD
or
(SEQ ID NO. 19)
GYIFTTKWIN.

14. The antibody or fragment according to claim 8 or 13, wherein said CDR H2 is defined as:
(SEQ ID NO. 12)
EIRLKSDNYATHYAESVKG
or
(SEQ ID NO. 20)
NIYPGSSNTYYNEKFKN.

15. The antibody or fragment according to any one of claims 8, 13 and 14, wherein said CDR H3 is defined as: APHYYGYFDY (SEQ ID NO.13) or GIAN (SEQ ID no. 21).

16. The antibody or fragment thereof of claim 1, comprising a peptide sequence:
comprising CDR sequences L1-3 defined by SEQ ID NO.7, 8 and 9; and
comprising CDR sequences H1-3 defined by SEQ ID NO.11, 12 and 13;
wherein said antibody binds CA-IX.

17. The antibody of claim 16, comprising a sequence defined by SEQ ID NO.6 and SEQ ID NO.10.

18. The antibody of claim 16, comprising a peptide sequence comprising SEQ ID NO.22 and SEQ ID NO.23.

19. The antibody or fragment of claim 4, comprising a peptide sequence:
comprising CDR sequences L1-3 defined by SEQ ID NO.15, 16 and 17; and
comprising CDR sequences H1-3 defined by SEQ ID NO. 19, 20 and 21;
wherein said antibody binds CA-IX.

20. The antibody of claim 19, comprising a peptide sequence defined by SEQ ID NO.14 and SEQ ID NO.18.

21. The antibody of claim 19, comprising peptide sequences defined by SEQ ID NO. 24 and SEQ ID NO.25.

22. A composition comprising the antibody as defined in any one of claims 16-18, in combination with the antibody as defined in any one of claims 19-21.

23. The antibody or fragment of any one of claims 1 to 22, wherein the antibody or fragment thereof is a full-length IgG, Fv, scFv, Fab, or F(ab′)₂.

24. The antibody or fragment of any one of claims 1 to 23, wherein the antibody or fragment thereof comprises framework regions from IgA, IgD, IgE, IgG, or IgM.

25. The antibody or fragment of any one of claims 1 to 24, wherein the antibody or fragment thereof is chimeric.

26. The antibody or fragment of claim 25, wherein the chimeric antibody or fragment thereof comprises a constant domain from human IgG2.

27. The antibody or fragment of claim 25, wherein the chimeric antibody or fragment thereof comprises human kappa-1 light chain and human IgG2 heavy chain constant domains.

28. A nucleic acid molecule encoding the antibody or fragment thereof according to any one of claims 1 to 27.

29. The nucleic acid of claim 28, comprising a sequence selected from the group consisting of: SEQ ID NO.38, 39, 40 and 41.

30. A vector comprising the nucleic acid molecule of claim 28 or 29.

31. A composition comprising one or more than one antibody or fragment as defined in any one of claims 1 to 27, in admixture with a pharmaceutically acceptable carrier, diluent, or excipient.

32. A method for detecting expression of Carbonic anhydrase-IX (CA-IX) in a biological sample, comprising the steps of:
a) contacting the biological sample with the antibody or fragment thereof according to any one of claims 1 to 27, wherein said antibody or fragment thereof is linked to a detectable agent; and
b) detecting the detectable agent linked to the antibody or fragment thereof bound to CA-IX in the biological sample.

33. The method of claim 32, wherein the steps are carried in vivo in a subject, and the biological sample is: blood or an organ.

34. The method of claim 33, wherein the organ is kidney.

35. The method of claim 33, wherein the subject is a human or an animal.

36. The method of claim 32, wherein the steps are carried in vitro, and the biological sample is from: blood, serum, urine or a biopsy tissue

37. The method of claim 36, wherein the biopsy tissue is a tumor or kidney tissue.

38. The method of any one of claims 32 to 37, wherein the step of detecting step b) is performed using: optical imaging, immunohistochemistry, molecular diagnostic imaging, ELISA, or other suitable method.

39. A method for inhibiting Carbonic anhydrase-IX (CA-IX) enzymatic activity in a cell, comprising contacting the cell with the antibody or fragment thereof as defined in any one of claims 1 to 27.

40. The method of claim 39, wherein the antibody or fragment thereof is linked to a chemotherapeutic drug.

41. A method of preventing or treating cancer in a subject, comprising administering a pharmaceutically acceptable dose of an antibody or fragment thereof as defined in any one of claims 1 to 27 to the subject.

42. Use of the antibody or fragment as defined in any one of claims 1 to 27, as a companion diagnostic in the adjuvant treatment of cancer.

43. The use of claim 42, wherein said cancer is Clear Cell Renal Cell Carcinoma (CCRCC).

44. Use of the antibody or fragment as defined in any one of claims 1 to 27, for the manufacture of a composition for the treatment or prevention of cancer in a subject.

45. The use of claim 44, wherein said cancer is Clear Cell Renal Cell Carcinoma (CCRCC).

46. The use of claim 44, wherein the subject is a human or an animal.

47. The antibody or fragment thereof according to any one of claims 1 to 27, for use in the treatment or prevention of cancer in a subject.

48. The antibody for use of claim 47, wherein said cancer is Clear Cell Renal Cell Carcinoma (CCRCC).

49. The antibody for use of claim 47, wherein the subject is a human or an animal.

50. An antigen-binding molecule that specifically binds to a 6 to 12 amino acid peptide comprised within the epitope STAFARVDEALGR (SEQ ID NO.5).

51. The antigen-binding molecule of claim 50, that specifically binds to a peptide comprising a sequence selected from the group consisting: DEALGR (SEQ ID NO.3) and STAFARVDE (SEQ ID NO.4).

52. The antigen-binding molecule of claim 51, that specifically binds to a peptide selected from the group consisting: DEALGR (SEQ ID NO.3) and STAFARVDE (SEQ ID NO.4).


SEQUENCE LISTING (201 sequences)

SEQ ID NO: 1 - protein, 121 aa, Artificial sequence
  Humanized, VH region
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSS

SEQ ID NO: 2 - protein, 8 aa, Artificial sequence
  VH region CDR1
GFTFTDFY

SEQ ID NO: 3 - protein, 10 aa, Artificial sequence
  VH region CDR2
IRDKAKGYTT

SEQ ID NO: 4 - protein, 12 aa, Artificial sequence
  VH region CDR2
AREGHTAAPFDY

SEQ ID NO: 5 - protein, 107 aa, Artificial sequence
  Humanized VL region
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIK

SEQ ID NO: 6 - protein, 6 aa, Artificial sequence
  VL region CDR1
QNIDKY

SEQ ID NO: 7 - protein, 9 aa, Artificial sequence
  VL region CDR3
LQHISRPRT

SEQ ID NO: 8 - protein, 125 aa, Artificial sequence
  VH region
EVQLVQSGGGLVHPGGSLRLSCTGSGFTFSYHAMHWVRQAPGKGLEWVSIIGTGGVTYYADSVKGRFTISRDNVKNSLYLQMNSLRAEDMAVYYCARDYYGAGSFYDGLYGMDVWGQGTTVTVSS

SEQ ID NO: 9 - protein, 8 aa, Artificial sequence
  VH region CDR1
GFTFSYHA

SEQ ID NO: 10 - protein, 7 aa, Artificial sequence
  VH region CDR2
IGTGGVT

SEQ ID NO: 11 - protein, 19 aa, Artificial sequence
  VH region CDR3
ARDYYGAGSFYDGLYGMDV

SEQ ID NO: 12 - protein, 107 aa, Artificial sequence
  VL region
EIVLTQSPATLSLSPGERATLSCRASQSVSSYLAWYQQKPGQAPRLLIYDASNRATGIPARFSGSGSGTDFTLTISSLEPEDFAVYYCQQRSDWPLTFGGGTKVEIK

SEQ ID NO: 13 - protein, 6 aa, Artificial sequence
  VL region CDR1
QSVSSY

SEQ ID NO: 14 - protein, 9 aa, Artificial sequence
  VL region CDR3
QQRSDWPLT

SEQ ID NO: 15 - protein, 127 aa, Artificial sequence
  VH region
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 16 - protein, 8 aa, Artificial sequence
  VH region CDR1
GYRFSNFV

SEQ ID NO: 17 - protein, 8 aa, Artificial sequence
  VH region CDR2
INPYNGNK

SEQ ID NO: 18 - protein, 20 aa, Artificial sequence
  VH region CDR3
ARVGPYSWDDSPQDNYYMDV

SEQ ID NO: 19 - protein, 108 aa, Artificial sequence
  VL region
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 20 - protein, 7 aa, Artificial sequence
  VL region CDR1
HSIRSRR

SEQ ID NO: 21 - protein, 9 aa, Artificial sequence
  VL region CDR1
QVYGASSYT

SEQ ID NO: 22 - protein, 330 aa, Human
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 330 aa, Human
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 330 aa, Human
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKPVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 330 aa, Human
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKPVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 26 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 27 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPGK

SEQ ID NO: 30 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKKLSLSPGK

SEQ ID NO: 31 - protein, 326 aa, Human
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 377 aa, Human
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 33 - protein, 327 aa, Human
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 107 aa, Human [sequence omitted]

SEQ ID NO: 35 - protein, 122 aa, Artificial sequence
  VH region
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 36 - protein, 8 aa, Artificial sequence
  VH region CDR1
GFTFHDYA

SEQ ID NO: 37 - protein, 8 aa, Artificial sequence
  VH region CDR2
ISWNSGTI

SEQ ID NO: 38 - protein, 15 aa, Artificial Sequence
  VH region CDR3
AKDIQYGNYYYGMDV

SEQ ID NO: 39 - protein, 107 aa, Artificial sequence
  VL region
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 40 - protein, 6 aa, Artificial sequence
  VL region CDR1
QSVSSY

SEQ ID NO: 41 - protein, 9 aa, Artificial sequence
  VL region CDR3
QQRSNWPIT

SEQ ID NO: 42 - protein, 115 aa, Artificial sequence
  VH region
QVQVKESGPGLVAPSQSLSITCTVSGFSLTTSGVSWVRQPPGKGLEWLGVIWGDGSTNYHSALKSRLSIKKDHSKSQVFLKLNSLQTDDTATYYCAKGGYSLAHWGQGTLVTVSA

SEQ ID NO: 43 - protein, 8 aa, Artificial sequence
  VH region CDR1
GFSLTTSG

SEQ ID NO: 44 - protein, 7 aa, Artificial sequence
  VH region CDR2
IWGDGST

SEQ ID NO: 45 - protein, 9 aa, Artificial sequence
  VH region CDR3
AKGGYSLAH

SEQ ID NO: 46 - protein, 107 aa, Artificial sequence
  VL region
DIQMTQSPASLSVSVGETVTITCRASENIRSNLAWYQQKQGKSPQLLVNVATNLADGVPSRFSGSGSGTQYSLKINSLQSEDFGTYYCQHYWGTTWTFGGGTKLEIK

SEQ ID NO: 47 - protein, 6 aa, Artificial sequence
  VL region CDR1
ENIRSN

SEQ ID NO: 48 - protein, 9 aa, Artificial sequence
  VL region CDR1
QHYWGTTWT

SEQ ID NO: 49 - protein, 118 aa, Artificial sequence
  Humanized VH region
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSS

SEQ ID NO: 50 - protein, 8 aa, Artificial sequence
  Humanized VH region CDR1
GFNIKDTF

SEQ ID NO: 51 - protein, 8 aa, Artificial sequence
  Humanized VH region CDR2
IDPANTNT

SEQ ID NO: 52 - protein, 11 aa, Artificial sequence
  Humanized VH region CDR3
VRGLYTYYFDY

SEQ ID NO: 53 - protein, 107 aa, Artificial sequence
  Humanized VL region
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIK

SEQ ID NO: 54 - protein, 6 aa, Artificial sequence
  Humanized VL region CDR1
QSISNN

SEQ ID NO: 55 - protein, 9 aa, Artificial sequence
  Humanized VL region CDR3
QQGNSWPYT

SEQ ID NO: 56 - protein, 118 aa, Artificial sequence
  Humanized VH region
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSS

SEQ ID NO: 57 - protein, 8 aa, Artificial sequence
  Humanized VH region CDR1
GFNIKDTH

SEQ ID NO: 58 - protein, 8 aa, Artificial sequence
  Humanized, VH region CDR2
IDPANGNT

SEQ ID NO: 59 - protein, 11 aa, Artificial sequence
  Humanized, VH region CDR3
ARWGTNVYFAY

SEQ ID NO: 60 - protein, 106 aa, Artificial sequence
  Humanized, VL region
DIQLTQSPSSLSASVGDRVTITCSASSSVSYMYWYQQKPGKAPKPWIYRTSNLASGVPSRFSGSGSGTDFTLTISSLQPEDFATYYCQQYHSYPPTFGGGTKVEIK

SEQ ID NO: 61 - protein, 5 aa, Artificial sequence
  Humanized, VL region CDR1
SSVSY

SEQ ID NO: 62 - protein, 9 aa, Artificial sequence
  Humanized, VL region CDR3
QQYHSYPPT

SEQ ID NO: 63 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKKLPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 64 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRAPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPGK

SEQ ID NO: 65 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRAPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKKLSLSPGK

SEQ ID NO: 66 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPGK

SEQ ID NO: 67 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPGK

SEQ ID NO: 68 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPISKTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKKLSLSPGK

SEQ ID NO: 69 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRQPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPGK

SEQ ID NO: 70 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRQPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKKLSLSPGK

SEQ ID NO: 71 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 72 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPISKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 73 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPGK

SEQ ID NO: 74 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPGK

SEQ ID NO: 75 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRVPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPGK

SEQ ID NO: 76 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRVPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKKLSLSPGK

SEQ ID NO: 77 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRYPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPGK

SEQ ID NO: 78 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRYPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKKLSLSPGK

SEQ ID NO: 79 - protein, 330 aa, Artificial sequence; identical to SEQ ID NO: 26
  modified Fc region

SEQ ID NO: 80 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 81 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHKDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 82 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 83 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLKGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 84 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 85 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 86 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 87 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGQPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 88 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGRPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 89 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGTPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 90 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 91 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCEVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 92 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEALGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 93 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 94 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 95 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 96 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 97 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 98 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 99 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 100 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQESLSLSPGK

SEQ ID NO: 101 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 102 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 103 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 104 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 105 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 106 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 107 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGQPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 108 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGRPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 109 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCEVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 110 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 111 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPIAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 112 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 113 - protein, 330 aa, Artificial sequence; identical to SEQ ID NO: 101
  modified Fc region

SEQ ID NO: 114 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 115 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHNALHNHYTQESLSLSPGK

SEQ ID NO: 116 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHNALHNHYTQKKLSLSPGK

SEQ ID NO: 117 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHTALHNHYTQESLSLSPGK

SEQ ID NO: 118 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHTALHNHYTQKKLSLSPGK

SEQ ID NO: 119 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHVALHNHYTQESLSLSPGK

SEQ ID NO: 120 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHVALHNHYTQKKLSLSPGK

SEQ ID NO: 121 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHYALHNHYTQESLSLSPGK

SEQ ID NO: 122 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHYALHNHYTQKKLSLSPGK

SEQ ID NO: 123 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 124 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 125 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGQPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 126 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGRPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 127 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGTPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 128 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQESLSLSPGK

SEQ ID NO: 129 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGQPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQESLSLSPGK

SEQ ID NO: 130 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKKLSLSPGK

SEQ ID NO: 131 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGAPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKKLSLSPGK

SEQ ID NO: 132 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCEVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 133 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 134 - protein, 326 aa, Artificial sequence
  modified Fc region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHGALHNHYTQKSLSLSPGK

SEQ ID NO: 135 - protein, 326 aa, Artificial sequence
  modified Fc region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHGALHNHYTQESLSLSPGK

SEQ ID NO: 136 - protein, 326 aa, Artificial sequence
  modified Fc region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVARPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHGALHNHYTQESLSLSPGK

SEQ ID NO: 137 - protein, 326 aa, Artificial sequence
  modified Fc region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHGALHNHYTQKKLSLSPGK

SEQ ID NO: 138 - protein, 326 aa, Artificial sequence
  modified Fc region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPISKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHGALHNHYTQKKLSLSPGK

SEQ ID NO: 139 - protein, 377 aa, Artificial sequence
  modified Fc region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHGALHNRFTQKSLSLSPGK

SEQ ID NO: 140 - protein, 377 aa, Artificial sequence
  modified Fc region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHGALHNRFTQESLSLSPGK

SEQ ID NO: 141 - protein, 377 aa, Artificial sequence
  modified Fc region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLRGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHGALHNRFTQESLSLSPGK

SEQ ID NO: 142 - protein, 377 aa, Artificial sequence
  modified Fc region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHGALHNRFTQKKLSLSPGK

SEQ ID NO: 143 - protein, 377 aa, Artificial sequence
  modified Fc region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPISKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHGALHNRFTQKKLSLSPGK

SEQ ID NO: 144 - protein, 327 aa, Artificial sequence
  modified Fc region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHGALHNHYTQKSLSLSLGK

SEQ ID NO: 145 - protein, 327 aa, Artificial sequence
  modified Fc region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHGALHNHYTQESLSLSLGK

SEQ ID NO: 146 - protein, 327 aa, Artificial sequence
  modified Fc region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLRGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHGALHNHYTQESLSLSLGK

SEQ ID NO: 147 - protein, 327 aa, Artificial sequence
  modified Fc region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHGALHNHYTQKKLSLSLGK

SEQ ID NO: 148 - protein, 327 aa, Artificial sequence
  modified Fc region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSISKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHGALHNHYTQKKLSLSLGK

SEQ ID NO: 149 - protein, 298 aa, Artificial sequence
  modified Fc gamma receptor
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHHHHHHH

SEQ ID NO: 150 - protein, 418 aa, Artificial sequence
  modified Fc gamma receptor
METQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPVAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGPGSSSHHHHHHPGGGLNDIFEAQKIEWHE

SEQ ID NO: 151 - protein, 405 aa, Artificial sequence
  modified Fc gamma receptor
MVLSLLYLLTALPGILSAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPGSSSHHHHHHPGGGLNDIFEAQKIEWHE

SEQ ID NO: 152 - protein, 405 aa, Artificial sequence
  modified Fc gamma receptor
MVLSLLYLLTALPGILSAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGPGSSSHHHHHHPGGGLNDIFEAQKIEWHE

SEQ ID NO: 153 - protein, 410 aa, Artificial sequence
  modified Fc gamma receptor
MVLSLLYLLTALPGISTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGPSMGSSSPSEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGPSMGSSSPGPGSSSHHHHHHPGGGLNDIFEAQKIEWHE

SEQ ID NO: 154 - protein, 410 aa, Artificial sequence
  modified Fc gamma receptor
MVLSLLYLLTALPGISTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGPSMGSSSPSEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGPSMGSSSPGPGSSSHHHHHHPGGGLNDIFEAQKIEWHE

SEQ ID NO: 155 - protein, 280 aa, Artificial sequence
  modified Fc region
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSHHHHHH

SEQ ID NO: 156 - protein, 99 aa, Human [sequence omitted]

SEQ ID NO: 157 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKKLSLSPGK

SEQ ID NO: 158 - protein, 330 aa, Artificial sequence
  modified Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 159 - protein, 653 aa, Artificial sequence
  Modified Her2
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRHHHHHH

SEQ ID NO: 160 - protein, 113 aa, Artificial sequence
  humanized VH region
EVHLVESGGGLVQPGGSLRLSCAASGFTFSRYGMSWVRQAPGKGLELVAMMKTKGGRTYYPDSVKGRFTISRDNAKNSLYLQMNSLRAEDTAIYYCASDGYYWGQGTTVTVSS

SEQ ID NO: 161 - protein, 8 aa, Artificial sequence
  humanized VH region CDR1
GFTFSRYG

SEQ ID NO: 162 - protein, 8 aa, Artificial sequence
  humanized VH region CDR2
MKTKGGRT

SEQ ID NO: 163 - protein, 6 aa, Artificial sequence
  humanized VH region CDR3
ASDGYY

SEQ ID NO: 164 - protein, 111 aa, Artificial sequence
  humanized VL region
DVVMTQTPLSLSVTLGQPASISCKSSQSLLHSDGKTYLNWLQQRPGQSPRRLIYLVSKLDSGVPDRFSGSGSGTDFTLKISRVEAEDVGIYYCWQGTHLWTFGGGTKVEIK

SEQ ID NO: 165 - protein, 11 aa, Artificial sequence
  humanized VL region CDR1
QSLLHSDGKTY

SEQ ID NO: 166 - protein, 8 aa, Artificial sequence
  humanized VL region CDR3
WQGTHLWT

SEQ ID NO: 167 - protein, 121 aa, Artificial sequence
  humanized VH region
QVQLQQSGSELKKPGASVKVSCKASGFTFTNYGMNWVKQAPGQGLKWMGWINTYTREPTYADDFKGRFAFSLDTSVSTAYLQISSLKADDTAVYFCARDITAVVPTGFDYWGQGSLVTVSS

SEQ ID NO: 168 - protein, 8 aa, Artificial sequence
  VH region CDR1
GFTFTNYG

SEQ ID NO: 169 - protein, 8 aa, Artificial sequence
  humanized VH region CDR2
INTYTREP

SEQ ID NO: 170 - protein, 14 aa, Artificial sequence
  humanized VH region CDR3
ARDITAVVPTGFDY

SEQ ID NO: 171 - protein, 107 aa, Artificial sequence
  humanized VL region
DIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIK

SEQ ID NO: 172 - protein, 6 aa, Artificial sequence
  humanized VL region CDR1
ENIYSN

SEQ ID NO: 173 - protein, 9 aa, Artificial sequence
  humanized VL region CDR3
QHFWTTPWA

SEQ ID NO: 174 - protein, 120 aa, Artificial sequence
  VH region
QVQLKESGPALVKPTQTLTLTCTFSGFSLSTSGVGVGWIRQPPGKALEWLALIDWDDDKYYSTSLKTRLTISKDTSKNQVVLTMTNMDPVDTATYYCARSPRYRGAFDYWGQGTLVTVSS

SEQ ID NO: 175 - protein, 10 aa, Artificial sequence
  VH region CDR1
GFSLSTSGVG

SEQ ID NO: 176 - protein, 7 aa, Artificial sequence
  VH region CDR2
IDWDDDK

SEQ ID NO: 177 - protein, 12 aa, Artificial sequence
  VH region CDR3
ARSPRYRGAFDY

SEQ ID NO: 178 - protein, 108 aa, Artificial sequence
  VL region
DIVLTQPPSVSGAPGQRVTISCSGSESNIGNNYVQWYQQLPGTAPKLLIYDNNQRPSGVPDRFSGSKSGTSASLAITGLQSEDEADYYCQSYDMNVHVFGGGTKLTVL

SEQ ID NO: 179 - protein, 8 aa, Artificial sequence
  VL region CDR1
ESNIGNNY

SEQ ID NO: 180 - protein, 9 aa, Artificial sequence
  VL region CDR3
QSYDMNVHV

SEQ ID NO: 181 - protein, 119 aa, Artificial sequence
  humanized VH region
EVQLVESGGGLVKPGGSLRLSCAASGFTFSSYGMFWVRQAPGKGLEWVATISRYSRYIYYPDSVKGRFTISRDNAKNSLYLQMNSLRAEDTAVYYCARRPLYGSSPDYWGQGTLVTVSS

SEQ ID NO: 182 - protein, 8 aa, Artificial sequence
  humanized VH region CDR1
GFTFSSYG

SEQ ID NO: 183 - protein, 8 aa, Artificial sequence
  humanized VH region CDR2
ISRYSRYI

SEQ ID NO: 184 - protein, 12 aa, Artificial sequence
  humanized VH region CDR3
ARRPLYGSSPDY

SEQ ID NO: 185 - protein, 106 aa, Artificial sequence
  humanized VL region
EIVLTQSPATLSLSPGERATLSCSASSSVTYVHWYQQKPGQAPRLLIYDTSKLASGIPARFSGSGSGTDFTLTISSLEPEDFAVYYCFQGSGYPLTFGSGTKLEMR

SEQ ID NO: 186 - protein, 5 aa, Artificial sequence
  humanized VL region CDR1
SSVTY

SEQ ID NO: 187 - protein, 9 aa, Artificial sequence
  humanized VL region CDR3
FQGSGYPLT

SEQ ID NO: 188 - protein, 118 aa, Artificial sequence
  VH region
EVQLQESGPGLVKPSQTLSLTCSVTGYSITSGYYWHWIRQFPGNKLEWMGYISYSGFTNYKTSLINRISITHDTSENQFFLNLNSVTTEDTATYYCAGDRTGSWFAYWGQGTLVTVSS

SEQ ID NO: 189 - protein, 9 aa, Artificial sequence
  VH region CDR1
GYSITSGYY

SEQ ID NO: 190 - protein, 7 aa, Artificial sequence
  VH region CDR2
ISYSGFT

SEQ ID NO: 191 - protein, 11 aa, Artificial sequence
  VH region CDR3
AGDRTGSWFAY

SEQ ID NO: 192 - protein, 107 aa, Artificial sequence
  VL region
DIQVTQSPSSLSASLGERISLTCRTSQDISNYLNWFQQKPDGTFKRLIYATSSLDSGVPKRFSGSGSGSDYSLTISSLESEDFADYYCLQYASYPFTFGSGTKLEIK

SEQ ID NO: 193 - protein, 6 aa, Artificial sequence
  VL region CDR1
QDISNY

SEQ ID NO: 194 - protein, 9 aa, Artificial sequence
  VL region CDR3
LQYASYPFT

SEQ ID NO: 195 - protein, 119 aa, Artificial sequence
  humanized VH region
EVQLQQSGAELVKPGASVKLSCTASGFNIKDTYMHWVKQRPEQGLEWIGRIDPANGNTKYDPKFQGKATITADTSSNTAYLQLSSLTSEDTAVYYCAYYYVSNAWFTYWGQGTLVTVSA

SEQ ID NO: 196 - protein, 8 aa, Artificial sequence
  humanized VH region CDR1
GFNIKDTY

SEQ ID NO: 197 - protein, 8 aa, Artificial sequence
  humanized VH region CDR2
IDPANGNT

SEQ ID NO: 198 - protein, 12 aa, Artificial sequence
  VH region CDR3
AYYYVSNAWFTY

SEQ ID NO: 199 - protein, 106 aa, Artificial sequence
  humanized VL region
DIQMTQSPASLSVSVGETVTITCRASENIYSNLEWYQQKQGKSPQLLVYAATNLADGVPSRFSGSGSGTQYSLKINSLQSEDFGSYYCQHFWGTWTFGGGTKLEIK

SEQ ID NO: 200 - protein, 6 aa, Artificial sequence
  humanized VL region CDR1
ENIYSN

SEQ ID NO: 201 - protein, 8 aa, Artificial sequence
  humanized VL region CDR3
QHFWGTWT